SEQUENCE LISTING (8 sequences)

SEQ ID NO: 1 - protein, 775 aa, Homo sapiens [sequence omitted]

SEQ ID NO: 2 - protein, 742 aa, Homo sapiens
TADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGPLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHY

SEQ ID NO: 3 - protein, 775 aa, Mus musculus
MAPAGHILTLLLWGHLLELWTPGHSANPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRRFRRQDVRHGNAAQQCFGQQFVGDALDRTEERLAYGIESNSTLLECTPRSLQAKVIWFVQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHFRLPRHTLLS

SEQ ID NO: 4 - protein, 741 aa, Mus musculus
ANPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRRFRRQDVRHGNAAQQCFGQQFVGDALDRTEERLAYGIESNSTLLECTPRSLQAKVIWFVQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHF

SEQ ID NO: 5 - protein, 775 aa, Rattus norvegicus
MAPTGHILTLLLWGHLLELWTPGYTANPSYPRLRLSHKELLELNRTSIFQSPFGFLDLHTMLLDEYQERLFVGGRDLVYSLNLERVSDGYREIYWPSTAVKVEECIMKGKDANECANYVRVLHHYNRTHLLTCATGAFDPHCAFIRVGHHSEDPLFHLESHRSERGRGRCPFDPNSSFVSTLVGNELFAGLYSDYWGRDSAIFRSLGKLGHIRTEHDDERLLKEPKFVGSYMIPDNEDPEDNKMYFFFTEKALEAENNAHTIYTRVGRLCVNDMGGQRILVNKWSTFLKARLVCSVPGMNGIDTHFDELEDVFLLPTRDPKNPVIFGLFNTTSNIFRGHAVCVYHMSSIREAFNGPYAHKEGPEYHWSLYEGKVPYPRPGSCASKVNGGKYGSTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHSKRRFRRQDVRHGNAAQQCFGQQFVGDALDRTEERLAYGIESNSTLLECTPRSLQAKVVWFVQKGRDVRKEEVKTDDRVVKMDLGLLFLRVRKSDAGTYFCQTVEHSFVHTVRKITLEVVEEHKVEGMFHKDREEEKHHKMPCPPLTGMSQGTKPWYKEFLQLIGYSNFQRVEEYCEKVWCTDKKRKKLKMSPSKWKYANPQEKRLRSKAEHFRLPRHTLVS

SEQ ID NO: 6 - protein, 741 aa, Rattus norvegicus
ANPSYPRLRLSHKELLELNRTSIFQSPFGFLDLHTMLLDEYQERLFVGGRDLVYSLNLERVSDGYREIYWPSTAVKVEECIMKGKDANECANYVRVLHHYNRTHLLTCATGAFDPHCAFIRVGHHSEDPLFHLESHRSERGRGRCPFDPNSSFVSTLVGNELFAGLYSDYWGRDSAIFRSLGKLGHIRTEHDDERLLKEPKFVGSYMIPDNEDPEDNKMYFFFTEKALEAENNAHTIYTRVGRLCVNDMGGQRILVNKWSTFLKARLVCSVPGMNGIDTHFDELEDVFLLPTRDPKNPVIFGLFNTTSNIFRGHAVCVYHMSSIREAFNGPYAHKEGPEYHWSLYEGKVPYPRPGSCASKVNGGKYGSTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHSKRRFRRQDVRHGNAAQQCFGQQFVGDALDRTEERLAYGIESNSTLLECTPRSLQAKVVWFVQKGRDVRKEEVKTDDRVVKMDLGLLFLRVRKSDAGTYFCQTVEHSFVHTVRKITLEVVEEHKVEGMFHKDREEEKHHKMPCPPLTGMSQGTKPWYKEFLQLIGYSNFQRVEEYCEKVWCTDKKRKKLKMSPSKWKYANPQEKRLRSKAEHF

SEQ ID NO: 7 - protein, 6 aa, unknown
  consensus site within sema3E polypeptide
KRRXRR

SEQ ID NO: 8 - protein, 4 aa, unknown
  consensus site within sema3E polypeptide
RXXR

(19) United States
(12) Patent Application Publication
(10) Pub. No.: US 2014/0066360 A1
(43) Pub. Date: Mar. 6, 2014

(54) METHODS FOR USING SEMAPHORIN POLYPEPTIDES

(71) Applicant: Abdelilah Soussi Gounni, Winnipeg (CA)

(72) Inventor: Abdelilah Soussi Gounni, Winnipeg (CA)

(73) Assignee: UNIVERSITY OF MANITOBA, Winnipeg, MB (CA)

(21) Appl. No.: 14/007,680

(22) PCT Filed: Mar. 28, 2012
(86) PCT No.: PCT/IB2012/000627

§ 371 Date: Nov. 11, 2013

Related U.S. Application Data
(60) Provisional application No. 61/469,441, filed on Mar. 30, 2011.
(60) Provisional application No. 61/554,075, filed on Nov. 1, 2011.

(51) Int. Cl. A61K 38/17; G01N 33/68

(52) U.S. Cl. CPC A61K 38/1703; G01N 33/6893
USPC 514/1.7; 435/7.1

(57) ABSTRACT

The present invention provides uses and methods of using a sema3E polypeptide and a sema3e polynucleotide encoding a sema3E polypeptide. The methods include decreasing airway remodeling in a subject, treating asthma in a subject, treating a subject having, or at risk of having, acute asthma, and reducing inflammation of a subject's airway. The methods also include evaluating treatment options for a subject having asthma and diagnosing whether a subject has asthma.

### CROSS-REFERENCE TO RELATED APPLICATIONS

This application claims the benefit of U.S. Provisional Application Ser. No. 61/469,441, filed Mar. 30, 2011, and U.S. Provisional Application Ser. No. 61/554,075, filed Nov. 1, 2011, each of which is incorporated by reference herein.

### BACKGROUND

Asthma is a chronic inflammatory disorder of the airways structurally characterized by bronchial hyperresponsiveness and airway remodeling. Increased airway smooth muscle (ASM) mass as the hallmark of airway remodeling might be the consequence of both ASM cell proliferation and migration (Bentley and Hershenson, Proc Am Thorac Soc, 2008, 5(1):89-96; Davies, D. E., et al., J Allergy Clin Immunol, 2003. 111(2):215-25; quiz 226; Halwani, R., et al., Curr Opin Pharmacol, 2010. 10(3):236-45; Mauad, T., et al., J Allergy Clin Immunol, 2007. 120(5):997-1009; quiz 1010-1).

Proliferation of ASM cells is increased in response to some growth factors and inflammatory mediators as well as allergen challenge. For instance, mitogenic effect of platelet-derived growth factor (PDGF) (Seidel, P., et al., Respir Res, 2010. 11:145, Simeone-Penney, M. C., et al., Am J Physiol Lung Cell Mol Physiol, 2008. 294(4):L698-704, Walker, T. R., et al., Mol Pharmacol, 1998. 54(6):1007-15), epidermal growth factor (EGF) (Cerutis, D. R., et al., Am J Physiol, 1997. 273(1 Pt 1):L10-5, Enomoto, Y., et al., J Allergy Clin Immunol, 2009. 124(5):913-20 e1-7), leukotriene B4 (Watanabe, S., et al., J Allergy Clin Immunol, 2009. 124(1):59-65 e1-3) and ovalbumin (Eynott, P. R., et al., Br J Pharmacol, 2003. 140(8):1373-80) on ASM cells has been previously demonstrated. More interestingly, there is a dramatic increase in proliferation rate of ASM cells obtained from asthmatic patients compared to non-asthmatic subjects in a time dependent manner (Johnson, P. R., et al., Am J Respir Crit Care Med, 2001. 164(3):474-7).

Increased accumulation of ASM cells in asthma is not solely because of ASM cell proliferation and it might be in part due to migration of ASM cell progenitors from outside the muscle toward the lumen or immigration of proliferating cells within the muscle bundles (Gerthoffer, Proc Am Thorac Soc, 2008. 5(1):97-105, Hirst, S. J., et al., J Allergy Clin Immunol, 2004. 114(2 Suppl):S2-17). Previous studies have demonstrated pro-migratory effect of various growth factors and inflammatory cytokines including PDGF, transforming growth factor (TGF) interleukin (IL)-1β and IL-8 on ASM cells (Govindaraju, V., et al., Am J Physiol Cell Physiol, 2006. 291(5):C957-65, Hedges, J. C., et al., J Biol Chem, 1999. 274(34):24211-9, Ito, I., et al., Clin Exp Allergy, 2009. 39(9):1370-80).

In contrast, some anti-asthmatic drugs including β₂-adrenergic receptor agonists and glucocorticoids have been shown to inhibit basal and growth factor-induced ASM cell proliferation and migration (Goncharova, E. A., et al., Am J Respir Cell Mol Biol, 2003. 29(1):19-27, Kassel, K. M., et al., Am J Physiol Lung Cell Mol Physiol, 2008. 294(1):L131-8, Stewart, A. G., et al., Mol Pharmacol, 1999. 56(5):1079-86, Stewart, A. G., et al., Br J Pharmacol, 1997. 121(3):361-8). However, the regulatory mechanisms underlying ASM cell proliferation and migration have not yet been clearly understood.

Originally discovered as axon guidance cues in neuronal development (Kolodkin, A. L., et al., Cell, 1993. 75(7):1389-99, Luo, Y., et al., Cell, 1993. 75(2):217-27), semaphorins are intrinsic versatile mediators involved in several aspects of cell functions including morphogenesis, angiogenesis, differentiation, cell proliferation and migration which are ubiquitously expressed in several tissues (Roth, L., et al., Cell Mol Life Sci, 2009. 66(4):649-66, Yazdani and Terman, Genome Biol, 2006. 7(3):211). Sema3E (SemaH or Coll-5), a vertebrate secreted semaphorin, was previously described as an endogenous mediator involved in axon path finding (Steinbach, K., et al., Exp Cell Res, 2002. 279(1):52-61) and vascular patterning (Adams and Eichmann, Cold Spring Harb Perspect Biol, 2010. 2(5):a001875, Gu, C., et al., Science, 2005. 307(5707):265-8). Besides, the Sema3E-PlexinD1 axis has also emerged as a pivotal mediator of cell migration, proliferation and angiogenesis in immune and endothelial contexts (Choi, Y. I., et al., Immunity, 2008. 29(6):888-98, Moriya, J., et al., Circ Res, 2010. 106(2):391-8).

### SUMMARY

The present invention provides use of a sema3E polypeptide. In one embodiment, the use is use of a sema3E polypeptide and a pharmaceutically acceptable carrier, in the preparation of a medicament for an inflammatory airway disease. In one embodiment, the use is use of a sema3E polypeptide and a pharmaceutically acceptable carrier, for treating an inflammatory airway disease. The inflammatory airway disease may be acute asthma and/or chronic asthma. The sema3E polypeptide may include a KRRXRR consensus site, wherein X is any amino acid. The sema3E polypeptide may further include one or two RXXR consensus sites, wherein X is any amino acid. In one embodiment, the sema3E polypeptide may include a Sema domain. In one embodiment, the sema3E polypeptide may further include one or more domains selected from a cystine rich domain, an immunoglobulin domain, and a short basic domain. In one embodiment, the sema3E polypeptide may further include a cystine rich domain and an immunoglobulin domain. In one embodiment, the sema3E polypeptide may include an amino acid sequence having at least 80% identity with SEQ ID NO:2. In one embodiment, the sema3E polypeptide is a fusion polypeptide. The sema3E polypeptide has activity, and the activity may be determined by ability to bind to Plexin D1, ability to inhibit airway smooth muscle cell migration or proliferation, or ability to influence development of airway hyper-reactivity during allergen challenge.

The present invention provides use of a sema3E polynucleotide. In one embodiment, the use is use of a sema3E polynucleotide and a pharmaceutically acceptable carrier, in the preparation of a medicament for an inflammatory airway disease, wherein the sema3E polynucleotide encodes a sema3E polypeptide. In one embodiment, the use is a use of a sema3E polynucleotide and a pharmaceutically acceptable carrier, for treating an inflammatory airway disease, wherein the sema3E polynucleotide encodes a sema3E polypeptide. The inflammatory airway disease may be acute asthma and/or chronic asthma. The sema3E polynucleotide may be present in a vector, such as a viral vector. The sema3E polypeptide may include a KRRXRR consensus site, wherein X is any amino acid. The sema3E polypeptide may further include one or two MOM consensus sites, wherein X is any amino acid. In one embodiment, the sema3E polypeptide may include a Sema domain. In one embodiment, the sema3E polypeptide may further include one or more domains selected from a cystine rich domain, an immunoglobulin domain, and a short basic domain. In one embodiment, the sema3E polypeptide may further include a cystine rich domain and an immunoglobulin domain. In one embodiment, the sema3E polypeptide may include an amino acid sequence having at least 80% identity with SEQ ID NO:2. In one embodiment, the sema3E polypeptide is a fusion polypeptide. The sema3E polypeptide has activity, and the activity may be determined by ability to bind to Plexin D1, ability to inhibit airway smooth muscle cell migration or proliferation, or ability to influence development of airway hyper-reactivity during allergen challenge.

The present invention also provides methods for using a sema3E polypeptide. In one embodiment, the method is for decreasing airway remodeling in a subject, including administering to a subject in need thereof an effective amount of a composition that includes a sema3E polypeptide, wherein the subject has decreased airway remodeling when compared to the subject before the administering. In one embodiment, the method is for treating asthma in a subject, including administering to the subject an effective amount of a composition that includes a sema3E polypeptide. In one embodiment, the method is for treating a subject having, or at risk of having, acute asthma, where the method includes administering to a subject in need thereof a composition that includes a sema3E polypeptide, wherein the subject has decreased airway resistance, decreased tissue resistance, decreased lung elastance, decreased eosinophilia in the bronchoalveolar space, or a combination thereof, when compared to the subject before the administering. In one embodiment, the method is for reducing inflammation of a subject's airway, where the method includes administering to a subject in need thereof a composition including sema3E polypeptide, wherein the subject has decreased inflammation of the airway when compared to the subject before the administering. In one embodiment the subject may be a human. In one embodiment the method may further include administering a therapeutic compound, such as an inhaled corticosteroid, an oral corticosteroid, a bronchodilator, a leukotriene antagonist, and/or an antihistamine. In one embodiment the subject has, or at risk of having, an inflammatory airway disease, such as acute asthma and/or chronic asthma.

In one embodiment, the method is for decreasing proliferation of a cell, where the method includes contacting an airway smooth muscle cell with an effective amount of a composition including a sema3E polypeptide. In one embodiment, the method is for decreasing migration of a cell, where the method includes contacting an airway smooth muscle cell with an effective amount of a composition that includes a sema3E polypeptide. In one embodiment the cell is a human cell. In one embodiment the cell is ex vivo, and in one embodiment the cell is in vivo.

The present invention also provides methods for using a sema3E polynucleotide. The polynucleotide may be present in a vector, such as a viral vector. In one embodiment, the method is for decreasing airway remodeling in a subject, where the method includes administering to a subject in need thereof an effective amount of a composition that includes a sema3E polynucleotide, wherein the subject has decreased airway remodeling when compared to the subject before the administering, and wherein the sema3E polynucleotide encodes a sema3E polypeptide. In one embodiment, the method is for treating asthma in a subject, where the method includes administering to the subject an effective amount of a composition that includes a sema3E polynucleotide, wherein the sema3E polynucleotide encodes a sema3E polypeptide. In one embodiment, the method is for treating a subject having, or at risk of having, acute asthma, where the method includes administering to a subject in need thereof a composition that includes sema3E polynucleotide, wherein the subject has decreased airway resistance, decreased tissue resistance, decreased lung elastance, decreased eosinophilia in the bronchoalveolar space, or a combination thereof, when compared to the subject before the administering, wherein the sema3E polynucleotide encodes a sema3E polypeptide. In one embodiment, the method is for reducing inflammation of a subject's airway, where the method includes administering to a subject in need thereof a composition that includes a sema3E polynucleotide, wherein the subject has decreased inflammation of the airway when compared to the subject before the administering, wherein the sema3E polynucleotide encodes a sema3E polypeptide. In one embodiment the subject may be a human. In one embodiment the method may further include administering a therapeutic compound, such as an inhaled corticosteroid, an oral corticosteroid, a bronchodilator, a leukotriene antagonist, and/or an antihistamine. In one embodiment the subject has, or at risk of having, an inflammatory airway disease, such as acute asthma and/or chronic asthma.

In one embodiment, the method is for decreasing proliferation of a cell, where the method includes contacting an airway smooth muscle cell with an effective amount of a composition that includes a sema3E polynucleotide, wherein the sema3E polynucleotide encodes a sema3E polypeptide. In one embodiment, the method is for decreasing migration of a cell, where the method includes contacting an airway smooth muscle cell with an effective amount of a composition that includes a sema3E polynucleotide, wherein the sema3E polynucleotide encodes a sema3E polypeptide. In one embodiment the cell is a human cell. In one embodiment the cell is ex vivo, and in one embodiment the cell is in vivo.

The present invention also provides a method for diagnosing whether a subject has asthma. In one embodiment the method includes obtaining a biological sample from the subject, wherein the biological sample includes an airway smooth muscle cell, measuring the expression of a sema3E polypeptide by the airway smooth muscle cell, and comparing the expression of a sema3E polypeptide by the airway smooth muscle cell with a control cell, wherein the presence of an airway smooth muscle cell that has reduced expression of a sema3E polypeptide compared to a control cell indicates the subject has asthma. The biological sample may include bronchial tissue, tracheal tissue, broncholaveolar lavage, sputum and/or serum. The method may further include administering to the subject a sema3E polypeptide or a fragment thereof. In one embodiment the subject is a human.

The present invention also provides a method for evaluating treatment options for a subject having asthma. In one embodiment the method includes obtaining a biological sample from the subject, wherein the biological sample includes an airway smooth muscle cell, measuring the expression of a sema3E polypeptide by the airway smooth muscle cell, and comparing the expression of a sema3E polypeptide by the airway smooth muscle cell with a control cell, wherein the presence of an airway smooth muscle cell that has reduced expression of a sema3E polypeptide compared to a control cell indicates the subject may be treated with a sema3E polypeptide. The biological sample may include bronchial tissue, tracheal tissue, broncholaveolar lavage, sputum and/or serum. The method may further include administering to the subject a sema3E polypeptide or a fragment thereof. In one embodiment the subject is a human.

The present invention also provides a method for diagnosing whether a subject has asthma. In one embodiment the method includes obtaining a biological sample from the subject, measuring the level of sema3E polypeptide in the biological sample, and comparing the level of sema3E polypeptide in the biological sample with the level of sema3E polypeptide in a control biological sample obtained from a healthy subject, wherein the presence of a decreased level of sema3E polypeptide compared to the control biological sample indicates the subject has asthma. The biological sample may include bronchial tissue, tracheal tissue, broncholaveolar lavage, sputum and/or serum. The method may further include administering to the subject a sema3E polypeptide or a fragment thereof. In one embodiment the subject is a human.

The present invention also provides a method for evaluating treatment options for a subject having asthma. In one embodiment the method includes obtaining a biological sample from the subject, measuring the level of sema3E polypeptide in the biological sample, and comparing the level of sema3E polypeptide in the biological sample with the level of sema3E polypeptide in a control biological sample obtained from a healthy subject, wherein the presence of a decreased level of sema3E polypeptide compared to the control biological sample indicates the subject may be treated with a sema3E polypeptide. The biological sample may include bronchial tissue, tracheal tissue, broncholaveolar lavage, sputum and/or serum. The method may further include administering to the subject a sema3E polypeptide or a fragment thereof. In one embodiment the subject is a human.

Also provided herein is a method for identifying an agent that increases sema3E polypeptide expression in a cell. The method includes contacting an airway smooth muscle cell with an agent, and measuring the expression of semaphorin 3E by the airway smooth muscle cell, wherein an increase in expression of semaphorin 3E in an airway smooth muscle cell compared to a control cell that was not contacted with the agent indicates the agent increases semaphorin 3E expression in a cell.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above description of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1: mRNA expression of Sema3E and its receptors in ASMCs. SEMA3E, PLXND1, NRP1 and VEGFR2 expression in four HASM cells was examined by RT-PCR analysis. cDNA was synthesized from total RNA, and amplified with specific primers. The RT-PCR products were separated on 2% agarose gels and stained with ethidium bromide.

FIG. 2: Protein expression of Sema3E and its receptors in ASMCs. Immunocytochemistry of Sema3E, PlexinD1, Nrp1 and VEGFR2 in HASM cells stained with relevant antibodies. Isotype control: cells stained without primary antibodies and with secondary antibodies.

FIG. 3: Immunoblot analysis of Sema3E expression in whole HASM cell lysate (L) and conditioned medium (CM). Medium was harvested and concentrated 50-fold using ultracentrifuge and also cells were lysed. CM and L were analyzed using gradient SDS-PAGE gels and human monoclonal anti-Sema3E antibody.

FIG. 4: Comparative immunohistochemistry for HASM cells derived from bronchial biopsies. Tissue sections were immuno-stained with anti-Sema3E polyclonal antibody, followed by secondary biotin conjugated antibody, counterstained with modified hematoxylin, and revealed by the AxioVision software using fast red as chromogen. Specimens A-D represent normal subjects, mild, moderate and severe asthmatics respectively, and E is isotype control.

FIG. 5: Effect of Sema3E on HASM cell migration. Migration of HASM cells following platelet derived growth factor (PDGF) stimulation was decreased when treated with Sema3E in a dose dependent manner. HASM cell migration in response to Sema3E was examined using a Boyden chamber. The values represent the average number of migrated cells±SEM. The graph is based on three independent migration experiments.

FIG. 6: HASM cell proliferation in response to Sema3E±PDGF. Proliferation of HASM cells was decreased when treated with Sema3E in a dose dependent manner. Four days after treatment, cells were collected and manually counted. The values represent the average number of counted cells±SEM. The graph is based on three independent proliferation experiments.

FIG. 7: HASM cell proliferation in response to Sema3E±PDGF. Human ASMCs were treated with different concentrations of Sema3E (1-1000 ng/mL) with or without PDGF and then specific proliferation was measured by EdU incorporation assay using flowcytometry (All data not shown).

FIG. 8: Actin reorganization after Sema3E stimulation in HASM cells. Phalloidin staining was performed to detect F-actin content of HASM cells following Sema3E treatment and results were quantified using flowcytometry as MFI.

FIG. 9. Schematic of schedule for inhalation of HDM for induction of asthma.

FIG. 10. Intranasal treatment with recombinant mouse Sema3E hampered HDM-induced AHR in a magnitude comparable to naïve mice in the acute model of asthma. Airway resistance (A-B), tissue resistance (C-D) and tissue elastance (E-F) were measured in response to inhaled methacholine and compared between two mouse strains with different backgrounds (A, C and E: Balb/c) (B, D and F: C57). Each graph, X-axis is methacholine dose (mg/ml), and Y-axis is cmH₂O/ml.

FIG. 11. Flow cytometric characteristics and cellular composition of BAL cells in HDM-challenged mice compared with naïve mice in both Balb/c and C57 strains. A and B (left columns): Distinction of Granulocytes (G), lymphocytes (L) and macrophages (M) based on the morphological flow cytometric parameters FSC and SSC. A-B (right columns): Ungated BAL cells in a CD3 vs. FSC plot. In Naïve (control) mice, mainly autofluorescent macrophages were recovered from the bronchoalveolar compartment. But, in asthmatic mice, granulocytes and lymphocytes were attracted and became the dominant populations in both Balb/c and C57 strains. Identification of BAL inflammatory cell populations using flow cytometry. To determine the major BAL cell types, surface marker staining was performed with fluorescently labeled antibodies regarding the following phenotypic patterns. CD3⁻: granulocytes, CD3⁺: lymphocytes or mononuclear cells (high-autofluorescent macrophages and non-autofluorescent DCs. Eosinophils: CD3⁻ CCR3⁺, Neutrophils: CD3⁻CCR3⁺, B cells: CD3⁻MHCII⁺, T cells: CD3⁺MHCII⁻, DCs: MHCII^{hi}/CD11c^{hi}, Macrophages: MHCII^{int/hi}/CD11c^{int}. C-D: Dominant macrophage population in naïve Balb/c mice with few granulocytes (C). This pattern is completely reversed in asthmatic Balb/c mice (D). E: Sema3E decreased granulocytes which were elevated in response to HDM (D) and also restored macrophages but not as high as control group (C). F-H: The same experiments and results from C57 model. SSC-A, side scatter; FSC-A, forward side scatter; Comp, compensation; APC-Cy7-A, APC-Cy7 is a tandem conjugate system that combines APC and a cyanine dye (Cy7); APC-Cy7-A::CD3, CD3 antibody conjugated with APC-Cy7; FITC-A, Fluorescein isothiocyanate; FITC-A::CD11c, CD11c antibody conjugated with FITC; PE-A::CCR3 antibody conjugated with phycoerythirn (PE); DCs, dendritic cells; Eos, eosinophil, and Neut, neutrophil.

FIG. 12. Comparison of flow cytometry and cytochemistry results. The percentage of eosinophils, neutrophils, lymphocytes and macrophages in the BAL of mice were determined by counting 200 cells on Giemsa stained cytospins using morphological criteria and compared with FACS data for all groups (Balb/c vs. C57 or naïve, asthmatic and Sema3E treated). Eos, eosinophil, Neut, neutrophil; Mono, monocyte; and Lymph, lymphocyte.

FIG. 13. A; Amino acid sequences of Semaphorin 3E from Homo sapiens, Mus musculus, and Rattus norvegicus. B; Multiple sequence alignment of SEQ ID NO:1, 3, and 5. “*” refers to identical amino acids in the consensus sequence; “:” refers to conserved amino acids in the consensus sequence.

### DETAILED DESCRIPTION OF ILLUSTRATIVE EMBODIMENTS

The present invention includes isolated polypeptides having semaphorin 3E activity. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, enzyme, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring. An “isolated” polypeptide is one that has been removed from a cell. For instance, an isolated polypeptide is a polypeptide that has been removed from the cytoplasm of a cell, and many of the polypeptides, nucleic acids, and other cellular material of its natural environment are no longer present. Polypeptides that are produced by recombinant, enzymatic, or chemical techniques are considered to be isolated and purified by definition, since they were never present in a cell. A “purified” polypeptide is one that is at least 60% free, preferably at least 75% free, and most preferably at least 90% free from other components of a cell.

Whether a polypeptide has semaphorin 3E activity may be determined by in vitro or in vivo assays. In one embodiment, semaphorin 3E activity refers to the ability of a polypeptide to bind Plexin D1 with an apparent K_{D} of <2 nM in a functional ELISA. For instance, when recombinant human Plexin D1 (such as catalog number 4160-PD, R&D Systems, Minneapolis, Minn.) is coated at 5 μg/mL, a polypeptide having semaphorin 3E activity will bind with an apparent K_{D} of <2 nM.

In one embodiment, semaphorin 3E activity refers to the ability of a polypeptide to inhibit basal and/or growth factor-induced airway smooth muscle cell migration and/or proliferation in a dose dependent manner. Methods for assaying the effect of a polypeptide on airway smooth muscle cell migration and/or proliferation is described in Example 1. A polypeptide is considered to have semaphorin 3E activity if there is a statistically significant decrease in basal and/or growth factor-induced airway smooth muscle cell migration and/or proliferation compared to control airway smooth muscle cells not exposed to the polypeptide.

In one embodiment, semaphorin 3E activity refers to the ability of a polypeptide to influence the development of airway hyper-reactivity during allergen challenge. Methods for evaluating the ability of a polypeptide to influence the development of airway hyper-reactivity during allergen challenge are described in Example 2. Examples of airway hyperreactivity include airway resistance, tissue resistance, and/or lung elastance. A polypeptide is considered to have semaphorin 3E activity if there is a statistically significant decrease in the development of airway hyper-reactivity as during allergen challenge in an animal, such as a mouse, compared to a control animal not exposed to the polypeptide.

A polypeptide having semaphorin 3E activity is referred to herein as a sema3E polypeptide. An example of a sema3E polypeptide is depicted at SEQ ID NO:2, which is amino acids 25-766 of SEQ ID NO:1 (SEQ ID NO:1 is available through the Genbank database at accession number AAI44339.1). Another example of a sema3E polypeptide is depicted at SEQ ID NO:4, which is amino acids 26-766 of SEQ ID NO:3 (SEQ ID NO:3 is available through the Genbank database at accession number NP_{—}035478.2). Another example of a sema3E polypeptide is depicted at SEQ ID NO:6, which is amino acids 26-766 of SEQ ID NO:5 (SEQ ID NO:5 is available through the Genbank database at accession number NP_{—}001100049.1). Other examples of sema3E polypeptides include amino acids 25-775 of SEQ ID NO:1, amino acids 26-775 of SEQ ID NO:3, or amino acids 26-775 of SEQ ID NO:5.

In one embodiment a sema3E polypeptide appears as an 87 kDa polypeptide on SDS-PAGE under reducing conditions. In one embodiment a sema3E polypeptide has an apparent molecular weight of 61 kDa or 25 kDa polypeptide on SDS-PAGE under reducing conditions. In one embodiment, a sema3E polypeptide migrates under non-reducing conditions as an oligomer with two 61 kDa polypeptides. In one embodiment, a sema3E polypeptide migrates under non-reducing conditions as an oligomer with one 61 kDa polypeptide and one 25 kDa polypeptide.

Other examples of sema3E polypeptides of the present invention include those that are structurally similar to the amino acid sequence of SEQ ID NO:2, 4, or 6. Structural similarity of two polypeptides can be determined by aligning the residues of the two polypeptides (for example, a candidate polypeptide and a reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference polypeptide may be a polypeptide described herein, such as SEQ ID NO:2, 4, or 6. A candidate polypeptide is the polypeptide being compared to the reference polypeptide. A candidate polypeptide may be isolated, for example, from a cell of an animal, such as a mouse, a rat, or a primate, such as a human, or can be produced using recombinant techniques, or chemically or enzymatically synthesized. A candidate polypeptide may be inferred from a nucleotide sequence present in the genome of an animal cell.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general parameters: expect threshold=10, word size=3, short queries=on; scoring parameters: matrix=BLOSUM62, gap costs=existence:11 extension:1, compositional adjustments=conditional compositional score matrix adjustment. Alternatively, polypeptides may be compared using the BESTFIT algorithm in the GCG package (version 10.2, Madison Wis.).

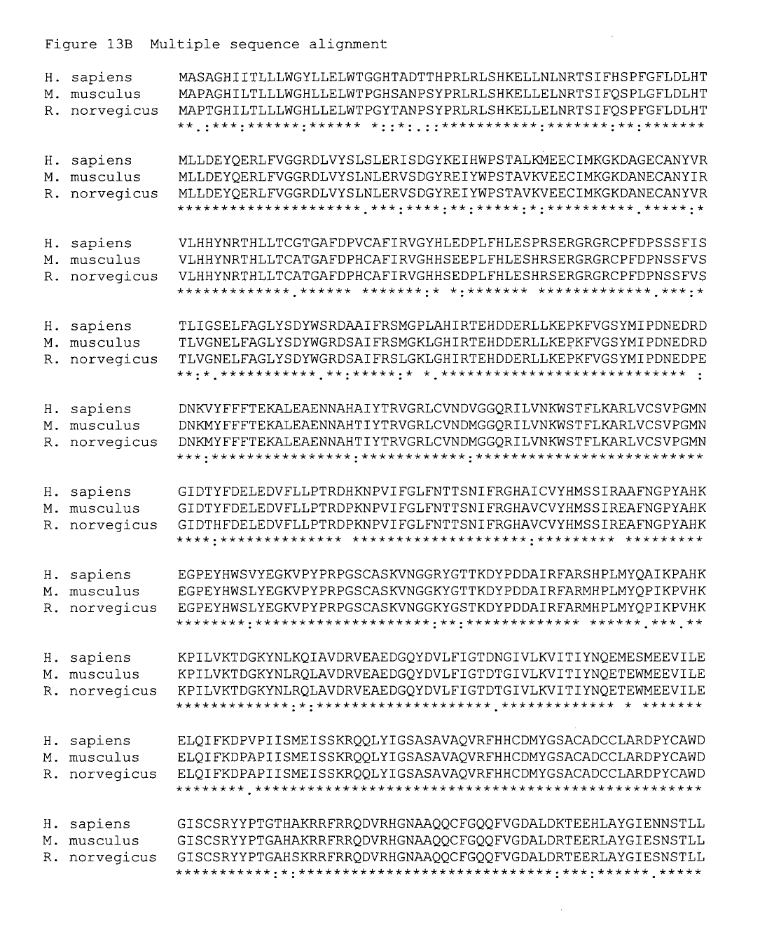

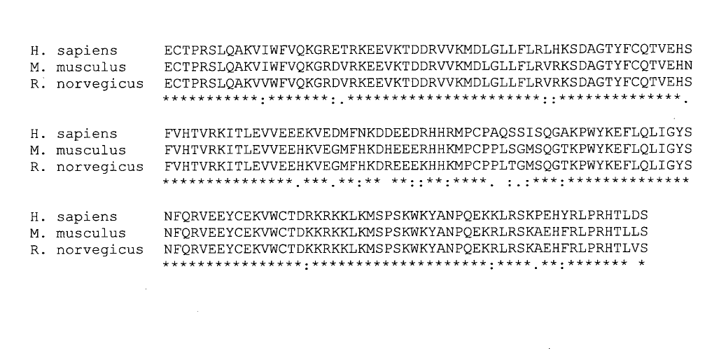

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. A high degree of identity is evident throughout the amino acid sequence of sema3E polypeptides. SEQ ID NO:1, 3, and 5 are shown in FIG. 13B in a multiple protein alignment. Identical and conserved amino acids are marked in the consensus sequence with “*” and “:”, respectively.

In one embodiment, a sema3E polypeptide includes consensus sites KRRXRR and RXXR, where X is any amino acid (Gherardi et al., 2004, Curr. Op. Struct. Biol., 14:669-678). In one embodiment, a sema3E polypeptide includes two MOM consensus sites, and in one embodiment, a sema3E polypeptide includes one RXXR consensus site, where the carboxy-terminal RXXR consensus site is not present. In one embodiment, a sema3E polypeptide includes consensus site KRRXRR. In one embodiment, a sema3E polypeptide includes amino acids corresponding to a Sema domain, a cystine rich domain, an Ig domain, and a short basic domain (Gherardi et al., 2004, Curr. Op. Struct. Biol., 14:669-678). In one embodiment, a sema3E polypeptide includes amino acids corresponding to a Sema domain, a cystine rich domain, and an immunoglobulin (Ig) domain.

Thus, as used herein, a sema3E polypeptide includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a sema3E polypeptide includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence.

A sema3E polypeptide having structural similarity the amino acid sequence of SEQ ID NO:2, 4, or 6 has semaphorin 3E activity. In one embodiment, a sema3E polypeptide may be a dimer.

The present invention also includes polypeptides having a length of less than SEQ ID NO:2, 4, or 6. For instance, a polypeptide of the present invention may include a sequence having a deletion of 1 or more amino acids from the amino terminal end, the carboxy terminal end, or a combination thereof, of SEQ ID NO:2, 4, or 6, or a polypeptide having structural similarity to SEQ ID NO: 2, 4, or 6. In one embodiment, a polypeptide may have a deletion of amino acids that includes or is greater than a number selected from at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues. In one embodiment, a polypeptide may have a deletion of amino acids that is no greater than a number selected from at least 1, at least of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues. In one embodiment, a polypeptide may have a deletion of number of amino acids selected from at least 1, at least of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues.

A polypeptide of the present invention may be expressed as a fusion that includes an additional amino acid sequence not normally or naturally associated with the polypeptide. In one embodiment, the additional amino acid sequence may be useful for purification of the fusion polypeptide by affinity chromatography. Various methods are available for the addition of such affinity purification moieties to proteins. Representative examples include, for instance, polyhistidine-tag (His-tag) and maltose-binding protein (see, for instance, Hopp et al. (U.S. Pat. No. 4,703,004), Hopp et al. (U.S. Pat. No. 4,782,137), Sgarlato (U.S. Pat. No. 5,935,824), and Sharma (U.S. Pat. No. 5,594,115)). In one embodiment, the additional amino acid sequence may be a carrier polypeptide. The carrier polypeptide may be used to increase the immunogenicity of the fusion polypeptide to increase production of antibodies that specifically bind to a polypeptide of the invention. The invention is not limited by the types of carrier polypeptides that may be used to create fusion polypeptides. Examples of carrier polypeptides include, but are not limited to, keyhole limpet hemacyanin, bovine serum albumin, ovalbumin, mouse serum albumin, rabbit serum albumin, and the like. In another embodiment, the additional amino acid sequence may be a fluorescent polypeptide (e.g., green, yellow, blue, or red fluorescent proteins) or other amino acid sequences that can be detected in a cell, for instance, a cultured cell, or a tissue sample that has been removed from an animal. If a polypeptide of the present invention includes an additional amino acid sequence not normally or naturally associated with the polypeptide, the additional amino acids are not considered when percent structural similarity to a reference amino acid sequence is determined.

Polypeptides of the present invention can be produced using recombinant DNA techniques, such as an expression vector present in a cell. Such methods are routine and known in the art. The polypeptides may also be synthesized in vitro, e.g., by solid phase peptide synthetic methods. The solid phase peptide synthetic methods are routine and known in the art. A polypeptide produced using recombinant techniques or by solid phase peptide synthetic methods can be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity. Such methods may also be used to isolate a polypeptide of the present invention from a cell.

The present invention also includes polynucleotides. In one embodiment, a polynucleotide encodes a polypeptide described herein. Also included are the complements of such polynucleotide sequences. A polynucleotide encoding a polypeptide having semaphorin 3E activity is referred to herein as a sema3E polynucleotide.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. Preferably, a polynucleotide of the present invention is isolated. An “isolated” polynucleotide is one that has been removed from a cell. For instance, an isolated polynucleotide is a polynucleotide that has been removed from a cell and many of the polypeptides, nucleic acids, and other cellular material of its natural environment are no longer present. Polynucleotides that are produced by recombinant, enzymatic, or chemical techniques are considered to be isolated and purified by definition, since they were never present in a cell.

Given the amino acid sequence of any one of the sema3E polypeptides described herein, a person of ordinary skill in the art can determine the full scope of polynucleotides that encode that amino acid sequence using conventional, routine methods. In one embodiment, a sema3E polynucleotide may have a nucleotide sequence encoding a polypeptide having the amino acid sequence shown at amino acids 25-766 of SEQ ID NO:1, amino acids 25-775 of SEQ ID NO:1, a polypeptide having sequence similarity with 25-766 of SEQ ID NO:1 or amino acids 25-775 of SEQ ID NO:1. It should be understood that a polynucleotide encoding a sema3E polypeptide represented by, for instance, amino acids 25-766 of SEQ ID NO:1 is not limited to a single nucleotide sequence, but includes the class of polynucleotides encoding such a polypeptide as a result of the degeneracy of the genetic code. For example, a naturally occurring nucleotide sequence encoding amino acids 25-766 of SEQ ID NO:1 is but one member of the class of nucleotide sequences encoding such a polypeptide. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A sema3E polynucleotide of the present invention may include heterologous nucleotides flanking the open reading frame encoding the semaphorin 3E polynucleotide. As used herein, “heterologous nucleotides” refers to a nucleotide sequence that is not normally or naturally found flanking a semaphorin 3E open reading frame in a cell. Typically, heterologous nucleotides may be at the 5′ end of the coding region, at the 3′ end of the coding region, or the combination thereof. Examples of heterologous nucleotides include, but are not limited to, a regulatory sequence. The number of heterologous nucleotides may be, for instance, at least 10, at least 100, or at least 1000.

A polynucleotide of the present invention can be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. Examples of viral vectors include, for instance, adenoviral vectors, adeno-associated viral vectors, lentiviral vectors, retroviral vectors, and herpes virus vectors. A vector may be replication-proficient or replication-deficient. A vector may result in integration into a cell's genomic DNA. Typically, a vector is capable of replication in a host cell, for instance a mammalian and/or a bacterial cell, such as E. coli.

Selection of a vector depends upon a variety of desired characteristics in the resulting construct, such as a selection marker, vector replication rate, use in gene transfer into cells of the respiratory tract, and the like. Suitable host cells for cloning or expressing the vectors herein are prokaryotic or eukaryotic cells. Suitable eukaryotic cells include mammalian cells, such as murine cells and human cells. Suitable prokaryotic cells include eubacteria, such as gram-negative organisms, for example, E. coli.

An expression vector optionally includes regulatory sequences operably linked to the polynucleotide of the present invention. An example of a regulatory sequence is a promoter. A promoter may be functional in a host cell used, for instance, in the construction and/or characterization of a Sema3E polynucleotide, and/or may be functional in the ultimate recipient of the vector. A promoter may be inducible, repressible, or constitutive, and examples of each type are known in the art. A polynucleotide of the present invention may also include a transcription terminator. Suitable transcription terminators are known in the art.

Polynucleotides of the present invention can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

The present invention is also directed to compositions including one or more polypeptides or polynucleotides described herein. Such compositions typically include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Additional active compounds can also be incorporated into the compositions.

A composition may be prepared by methods well known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a polypeptide or polynucleotide described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredients such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

Oral compositions may include an inert diluent or an edible carrier. For the purpose of oral therapeutic administration, the active compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds may be delivered in the form of an aerosol spray, a nebulizer, or an inhaler, such as a nasal spray, metered dose inhaler, or dry-powder inhaler.

Systemic administration can also be by transmucosal or transdermal means For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds may be formulated into ointments, salves, gels, or creams as generally known in the art. An example of transdermal administration includes iontophoretic delivery to the dermis or to other relevant tissues.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Delivery reagents such as lipids, cationic lipids, phospholipids, liposomes, and microencapsulation may also be used.

In one embodiment, an active compound may be associated with a targeting group. As used herein, a “targeting group” refers to a chemical species that interacts, either directly or indirectly, with the surface of a cell, for instance with a molecule present on the surface of a cell, e.g., a receptor. The interaction can be, for instance, an ionic bond, a hydrogen bond, a Van der Waals force, or a combination thereof. Examples of targeting groups include, for instance, saccharides, polypeptides (including hormones), polynucleotides, fatty acids, and catecholamines. Another example of a targeting group is an antibody. The interaction between the targeting group and a molecule present on the surface of a cell, e.g., a receptor, may result in the uptake of the targeting group and associated active compound.

When a polynucleotide is introduced into cells using a suitable technique, the polynucleotide may be delivered into the cells by, for example, transfection or transduction procedures. Transfection and transduction refer to the acquisition by a cell of new genetic material by incorporation of added polynucleotides. Transfection can occur by physical or chemical methods. Many transfection techniques are known to those of ordinary skill in the art including, without limitation, calcium phosphate DNA co-precipitation, DEAE-dextrin DNA transfection, electroporation, naked plasmid adsorption, cationic liposome-mediated transfection (commonly known as lipofection), use of glycoconjugates and polyplexes, targeting serpin-enzyme complex receptors, and polyethyleneimine. Transduction refers to the process of transferring nucleic acid into a cell using a DNA or RNA virus. Introduction of a polynucleotide into a cell may further include the use of methods to permeabilize the glycocalyx and epithelium of the respiratory tract, such as temporary water-induced hypoosmotic shock (Kolb et al., 2006, Chest, 130:879-884).

Toxicity and therapeutic efficacy of such active compounds can be determined by standard pharmaceutical procedures in cell cultures or experimental animals, e.g., for determining the ED₅₀ (the dose therapeutically effective in 50% of the population).

The data obtained from cell culture assays and animal studies can be used in formulating a range of dosage for use in humans. The dosage of such active compounds lies preferably within a range of concentrations that include the ED₅₀ with little or no toxicity. The dosage may vary within this range depending upon the dosage form employed and the route of administration utilized. For an active compound used in the methods of the invention, it may be possible to estimate the therapeutically effective dose initially from cell culture assays. A dose may be formulated in animal models to achieve a concentration range that includes the IC₅₀ (i.e., the concentration of the test compound which achieves a half-maximal inhibition of signs and/or symptoms) as determined in cell culture. Such information can be used to more accurately determine useful doses in humans.

The compositions can be administered one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of a polynucleotide or a polypeptide can include a single treatment or can include a series of treatments.

The present invention includes methods for using the polypeptides and polynucleotides disclosed herein. In one embodiment, a method includes contacting a cell with an effective amount of a sema3E polypeptide. In one embodiment, the contacting is under conditions suitable for allowing the sema3E polypeptide to interact with the surface of the cell. In one embodiment, the contacting is under conditions suitable for introduction of a sema3E polypeptide into the cell. In another embodiment, a method includes contacting a cell with an effective amount of a sema3E polynucleotide. In one embodiment, the contacting is under conditions suitable for introduction of a sema3E polynucleotide into the cell.

Conditions that are “suitable” for an event to occur, such as introduction of a polypeptide into a cell, or “suitable” conditions are conditions that do not prevent such events from occurring. Thus, these conditions permit, enhance, facilitate, and/or are conducive to the event. As used herein, an “effective amount” relates to a sufficient amount of a sema3E polypeptide or a sema3E polynucleotide to provide the desired effect. For instance, in one embodiment an “effective amount” is an amount effective to alter certain characteristics of cells. Examples of characteristics include, but are not limited to, proliferation and migration. The method may result in decreasing proliferation of the cell, decreasing migration of the cell, or the combination. In one embodiment, a cell is considered to have a decrease in proliferation or migration is if there is a statistically significant decrease in either proliferation or migration compared to a control not contacted with the sema3E polypeptide or the sema3E polynucleotide. In one embodiment, a cell is considered to have a decrease in proliferation or migration is if there is a decrease in proliferation or migration of at least 10%, at least 20%, at least 30%, at least 40%, at least 50%, at least 60%, at least 70%, or at least 80% compared to a control not contacted with the sema3E polypeptide or the sema3E polynucleotide.

A cell that may be used in the methods described herein may be ex vivo or in vivo. As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long term culture in tissue culture medium). Examples of primary cells include cells normally present in an animal's respiratory tract, including, but not limited to, airway smooth muscle cells (such as tracheal smooth muscle cells and bronchial smooth muscle cells), cells present in bronchoalveolar lavage, epithelial cells, mast cells, endothelial cells, and fibroblasts. Examples of cultured cells include, but are not limited to, epithelial cells, mast cells, endothelial cells, and fibroblasts. Control cells may be obtained from the ATCC and may be cultured according to methods known in the art. Control cells may also be obtained from tissue samples through, for example, biopsy. As used herein, “in vivo” refers to a cell that is present within an animal. A cell that may be used in the methods described herein may be a mammalian cell, such as, for instance, mouse, rat, primate (e.g., monkey, human), or horse.

The present invention also includes methods for treating certain diseases. In one embodiment, a method includes treating a disease in a subject, where a subject in need thereof is administered an effective amount of a composition that includes a sema3E polypeptide or a sema3E polynucleotide. The subject may be a mammal, such as a member of the family Muridae (a murine animal such as rat or mouse), a primate, (e.g., monkey, human), a dog, a sheep, a guinea pig, or a horse. As used herein, the term “disease” refers to any deviation from or interruption of the normal structure or function of a part, organ, or system, or combination thereof, of a subject that is manifested by a characteristic symptom or clinical sign. Diseases include respiratory conditions, such as inflammatory diseases of the airway. Examples of inflammatory diseases of the airway include, but are not limited to, fibrosis (including idiopathic pulmonary fibrosis and cystic fibrosis), asthma (including acute asthma and chronic asthma), chronic bronchitis, acute bronchitis, emphysema, and chronic obstructive pulmonary disease (COPD). The inflammatory diseases of the airway may have an environmental cause or a genetic cause, and may be exercise induced or occupational. Diseases also include inflammatory diseases of an organ, such as the kidney.

As used herein, the term “symptom” refers to subjective evidence of disease or condition experienced by the patient and caused by disease. As used herein, the term “clinical sign,” or simply “sign,” refers to objective evidence of a disease present in a subject. Symptoms and/or signs associated with diseases referred to herein and the evaluation of such signs are routine and known in the art. Examples of signs of disease may include, but are not limited to, wheezing, coughing, chest tightness, shortness of breath, reversible airflow obstruction, airway remodeling, bronchospasm, increased airway resistance, increased tissue resistance, increased lung elastance, increased inflammation of the airway, and increased airway inflammatory responses, such as increased eosinophilia in the bronchoalveolar space. A symptom and/or sign may be localized to, for instance, a subject's trachea, bronchi, bronchioles, alveoli, or a combination thereof. Whether a subject has a disease, and whether a subject is responding to treatment, may be determined by evaluation of signs associated with the disease.

In one embodiment, a disease treated using a method of the present invention is acute asthma. Signs of acute asthma may include, but are not limited to, increased airway resistance, increased tissue resistance, increased lung elastance, and increased eosinophilia in the bronchoalveolar space. In one embodiment, a disease treated using a method of the present invention is chronic asthma. Signs of chronic asthma that may be treated include, but are not limited to, hyperreactivity, bronchoconstriction, shortness of breath, and mucus secretion.

Treatment of a disease can be prophylactic or, alternatively, can be initiated after the development of a disease. Treatment that is prophylactic, for instance, initiated before a subject manifests signs of a disease, is referred to herein as treatment of a subject that is “at risk” of developing a disease. An example of a subject that is at risk of developing a disease is a person having a risk factor. Examples of risk factors include a history of atopic disease, a family history of asthma, airway hyperreactivity, and/or exposure to environmental conditions linked to certain respiratory conditions. Treatment can be performed before, during, or after the occurrence of the diseases described herein. Treatment initiated after the development of a disease may result in decreasing the severity of the signs of the disease, or completely removing the signs. An “effective amount” may be an amount effective to alleviate one or more symptoms and/or signs of the disease. In one embodiment, an effective amount is an amount that is sufficient to effect a reduction in a symptom and/or sign associated with a disease. A reduction in a symptom and/or a sign is, for instance, at least 10%, at least 20%, at least 30%, at least 40%, at least 50%, at least 60%, at least 70%, at least 80%, at least 90%, or at least 100% in a measured sign as compared to a control, a non-treated subject, or the subject prior to administration of the sema3E polypeptide or semaphorin 3E. It will be understood, however, that the total daily usage of the compositions and formulations as disclosed herein will be decided by the attending physician within the scope of sound medical judgment. The exact amount required will vary depending on factors such as the type of disease being treated.

The polypeptides and/or polynucleotides described herein may also be administered to a subject in combination with other therapeutic compounds to increase the overall therapeutic effect. Therapeutic compounds useful for the treatment of the diseases described herein are known and used routinely. Therapeutic compounds may include inhaled corticosteroids, for example beclomethasone, budesonide, fluticasone, or mometasone; oral corticosteroids, for example prednisone; bronchodilators, for example, beta-agonist bronchodilators such as albuterol, salmeterol, formoterol metaproterenol, pirbuterol, terbutaline, isoetharine, levalbuterol or salmetrol; leukotriene antagonists, for example montelukast sodium; and antihistamines, including for example, cetirizine, fexofenadine, loratadine, desloratadine, promethazine, alimemazine, dexchlorpheniramine, brompheniramine, buclizine, carbinoxamine and doxylamine.

The present invention also includes methods for diagnosing whether a subject has, or is at risk of having, asthma. The method may include measuring the expression of a sema3E polypeptide by an airway smooth muscle cell. A decrease of semaphorin 3E in the cell relative to a control cell indicates the subject has, or is at risk of having, asthma. Optionally, the method also includes obtaining a biological sample from the subject. Such a method may be used to evaluate treatment options for a subject having asthma. For instance, such a method may indicate that treatment with a sema3E polypeptide or a sema3E polynucleotide is appropriate.

As used herein, a “biological sample” refers to a sample of tissue or fluid isolated from a subject, including but not limited to, for example, cells, and tissues such as biopsy samples, from a respiratory tract, such as tracheal, bronchial cells and/or tissues, or fluids such as broncholaveolar lavage, sputum, or serum. Biological samples also include explants and primary and/or transformed cell cultures derived from patient tissues. A biological sample can be provided by removing a sample of cells or a fluid from a subject, but can also be accomplished by using previously isolated cells (e.g., isolated by another person, at another time, and/or for another purpose). Methods for measuring the amount of polypeptides such as semaphorin 3E are known in the art and are routine. Such methods include, for instance, Western immunoblot, ELISA, immunoprecipitation, or immunohistochemistry. Western immunoblot and immunoprecipitation are generally used with ex vivo cells, and immunohistochemistry is generally used with in vivo or ex vivo cells. Antibody to semaphorin 3E is commercially available.

In one embodiment, the methods include contacting cells of a subject's airway with an effective amount of a composition that includes a sema3E polypeptide or a sema3E polynucleotide. The method may result in decreasing migration of cells, such as eosinophils, into the bronchoalveolar space. The method may result in decreasing migration and/or maturation of cells, such as dendritic cells. In one embodiment, the subject may be suffering from, or at risk of suffering from, acute asthma.

The present invention also provides a method for identifying a compound that increases the amount of a Sema3E polypeptide in a cell, or promotes the activity of a Sema3E polypeptide (e.g., increases semaphorin 3E activity as determined using an in vitro or in vivo assay described herein) in a cell. The method includes contacting a cell, such as an airway smooth muscle cell, with a compound, incubating the cell and the compound under conditions suitable for culturing the cell, and measuring. The measuring may include, but is not limited to, assessing changes in ability to bind to plexin D1, and changes in basal and/or growth factor-induced airway smooth muscle cell migration and/or proliferation. The compound may be a chemical compound, including, for instance, an organic compound, an inorganic compound, a metal, a polypeptide, a non-ribosomal polypeptide, a polyketide, or a peptidomimetic compound. A compound can be obtained using any of the numerous approaches in combinatorial library methods known in the art, including biological libraries and synthetic library methods. The sources for potential compounds to be screened include, for instance, chemical compound libraries, cell extracts of plants and other vegetations.

The present invention is illustrated by the following examples. It is to be understood that the particular examples, materials, amounts, and procedures are to be interpreted broadly in accordance with the scope and spirit of the invention as set forth herein.

### Example 1

### Semaphorin 3E is Expressed in Human Airway Smooth Muscle Cells and Inhibits their Proliferation and Migration

Objectives: This example investigates the expression, functional role and signaling of Sema3E in human airway smooth muscle (HASM) cells.

Methods: RT-PCR, Immunocyto/histochemistry and Western blotting were used to determine the expression of Sema3E and its receptors. Cell proliferation was assessed by cell count and EdU incorporation assay and cell migration was evaluated using Boyden chamber assay.

Results: The data demonstrated expression of Sema3E and its receptors in HASM cells. We further showed differential expression of Semap3E in normal and asthmatic human bronchial specimens. Sema3E dramatically inhibited HASM cell proliferation and migration along with induction of F-actin depolymerization.

Conclusions: These data reveal for the first time that semaphorins play functional roles in HASM cells and proposes that semaphorins might contribute to airway remodeling in asthma.

There is no study demonstrating expression, function and signaling of semaphorins in ASM cells and their contribution to airway remodeling (pathogenesis of asthma). We investigated whether Sema3E and its holoreceptor components—including PlexinD1, Neuropilin 1 (Nrp1) and Vascular Endothelial Growth Factor 2 (VEGFR2)—are expressed in HASM cells. Then, we investigated Sema3E effects on HASM cell proliferation and migration. Furthermore, we sought the signaling pathways leading to Sema3E effects on HASM cell function.

### Methods

### Reagents:

Recombinant human Semaphorin 3E and PDGF-BB as well as human Semaphorin 3E, Plexin D1, Neuropilin-1 and VEGF R2/KDR/Flk-1 antibodies were purchased from R&D Systems (Minneapolis, Minn.). Mouse, rabbit and goat IgG1 isotype controls were from Sigma-Aldrich Canada (Oakville, Ontario, Canada). Cell culture media including Dulbecco's Modified Eagle Medium (DMEM) and F-12, antibiotics (penicillin and streptomycin were obtained from Invitrogen Canada (Burlington, Ontario, Canada); the FBS was obtained from HyClone Laboratories (Logan, Utah). Alkaline phosphatase-conjugated streptavidin was purchased from Jackson Immuno-Research Laboratories (West Grove, Pa.). Click it EdU Proliferation assay kits and Phalloidin Alexa-647 were obtained from Invitrogen (Burlington, Ontario, Canada).

### Isolation and Culture of HASM Cells:

Primary HASM cells used in this study were tracheal smooth muscle cells isolated from explants and bronchial smooth muscle cells obtained from macroscopically healthy segments of second and fourth generation lobar or main bronchus obtained after lung resection surgery of patients with adenocarcinoma. A HASM cell line immortalized by the stable expression of human telomerase reverse transcriptase (hTERT) was also used in some experiments. Cell culture condition has been described in our previous studies (Rahman, M. S., et al., J Immunol, 2006. 177(6):4064-71, Saleh, A., et al., J Immunol, 2009. 182(6):3357-65, Yamasaki, A., et al., PLoS One, 2010. 5(2):e9178, Zhang, K., et al., Am J Physiol Lung Cell Mol Physiol, 2007. 293(2):L375-82). The procedures were approved by the Ethics Committee of the University of Manitoba (Winnipeg, Manitoba, Canada).

### RNA Extraction and RT-PCR:

Total RNA was isolated from HASM cells using TRIzol method (Invitrogen, Canada) and its concentration was measured by nanodrop. Two micrograms of total RNA was subjected to MultiScribe™ Reverse Transcriptase to synthesize cDNA according to manufacturer's instructions (Applied Biosystems). Expression of SEMA3E, PLXND1, NRP1, VEGFR2 and GAPDH (as a housekeeping gene) at mRNA level was analyzed by RT-PCR. Human universal RNA was used as a positive control in all experiments.

### Immunocytochemistry:

HASM cells were seeded onto sterile uncoated glass coverslips and grown to 50-70% confluency. Cells were then fixed, permeabilized, and non-specific antibody binding was blocked with blocking buffer containing 5% normal human serum and 5% normal donkey serum in Tris-buffered saline (TBS) for 1 h at room temperature. Immunolabeling was performed using anti-human Sema3E, PlexinD1, Nrp1 or VEGFR2 antibodies as well as appropriate isotype negative control IgG at 4° C. overnight. Cells were incubated with streptavidin-alkaline phosphatase-conjugated secondary antibodies (Jackson Immuno Research Laboratories, West Grove, Pa.) for 1 h at room temperature. Except blocking, cells were extensively washed using cyto-TBS (pH 7.4) after each step. The signals were detected by addition of Fast-red (Sigma-Aldrich, Saint Loius, Mo.) and then cells were counterstained with Harris modified hematoxilin (Fisher Scientific, Fair Lawn, N.J.). Coverslips were mounted using crystal mount and visualized by AxioVision software (Carl Zeiss, Inc.).

### Immunoblot Blot Analysis:

HASM cells were grown in DMEM containing 10% FBS and serum deprived for 48 h in F12 medium containing Insulin-Transferrin-Selenium-X (ITS) supplement (Gibco) and sodium pyruvate. Conditioned medium was collected two days later, filtrated, and then concentrated 50× using centrifugal filter units (Amicon Ultra-4, Milipore). In parallel, HASM cells grown in the same medium were lysed using M-PER mammalian protein extraction reagent (Pierce) containing protease inhibitor cocktail (Roche, Germany). Total protein concentration was measured by Lowry method prior to Western blot experiments. Immunoblot analysis was performed using 2 μg/mL of monoclonal anti-human Sema3E antibody.

### Immunohistochemistry:

In vivo expression of Sema3E was evaluated by immunostaining of tissue sections obtained from bronchial biopsies of mild, moderate and severe allergic asthmatic subjects and normal individuals with procedures approved by the Human Research Ethics Board of the Laval University (Quebec, Canada). Subjects were of mixed gender between 18-40 years of age. Samples were categorized based on the following selection criteria. Normal: FEV1≧100% predicted, methacholine PC20>32 mg/mL; mild asthmatic: FEV1≧80-100% predicted, methacholine PC20<16 mg/mL; moderate to severe asthmatic: FEV1<80 predicted, methacholine PC20<2 mg/mL. Formalin-fixed paraffin-embedded sections were dewaxed with xylene and rehydrated in a gradient of 95% and 70% of ethanol to water and then boiled with microwave for 10 min in sodium citrate buffer (pH 6.0). After washing and blocking the sections, as mentioned earlier, goat anti-human Sema3E pAb or isotype control goat•IgG1 were added and sections were incubated overnight at 4° C. Addition of secondary antibody, development with Fast-red, counterstaining and visualization were the same as the immunocytochemistry experiments.

### Proliferation Assays:

### Cell Counting:

HASM cells were seeded at 5×10⁴ cells/well in triplicates in 6-well plates and maintained in DMEM to become 50% to 70% confluent. After serum starvation as described above, cells were treated with Sema3E (0, 1, 10 and 100 ng/mL) (R& D Systems, MN) with or without PDGF (10 ng/mL). Four days later, cells were collected and counted using a hemocytometer and cell viability was determined by trypan blue exclusion. Cell count experiments were performed double-blind and twice by two independent individuals.

### EdU Cell Proliferation Assay:

Serum deprived HASM cells were stimulated with Sema3E (0, 1, 10, 100 and 1000 ng/mL) with or without PDGF (10 ng/mL) Click-iT™ EdU flow cytometry assay kit (Invitrogen, Eugene, Oreg.) was used to evaluate Sema3E effects on HASM cell proliferation. Briefly, EdU reagent was added at a 10 μM final concentration 16 h after stimulation and cells were harvested 24 h later. Cells were collected into phosphate-buffered saline (PBS) containing 1% BSA, centrifuged fixed and then incubated with saponin-based permeabilization buffer for 15 min. Click-iT reaction cocktail was freshly prepared and added to the samples according to the manufacturer's instructions. EdU incorporation into newly synthesized DNA was assessed using flow cytometry (FACStar, Consort 30 System; Becton-Dickinson, Mountain View, Calif.).

### Cell Migration Assay:

Boyden chamber (Neuro Probe Inc. Gaithersburg, Md.) was employed to study HASM cell migration as described previously (Goncharova, E. A., et al., Nat Protoc, 2006. 1(6):2933-9). Sema3E (0, 0.1, 1 and 10 μg/mL) with or without PDGF (10 ng/mL) in F12 medium containing ITS and sodium pyruvate was added to the wells of the bottom chamber in triplicate. Then, Collagen-coated filter membrane was placed on the bottom chamber and serum-deprived HASM cells (5×10⁵ cell/mL) were added to the upper chamber wells. After 4 h of incubation in 37° C., non-migrated cells that did not pass through the membrane pores were wiped out from the top side of the insert membrane and then migrated cells in each well were stained with Protocol Hema 3 stain (Biochemical Sciences Inc. Swedesburg, N.J.) and counted in random fields (200).

### Immunoflourescence (Phalloidin Staining):

The cells cultured on coverslips were serum-starved and treated with Sema3E (100 ng/ml) for 30-120 min, fixed with 3.7% formaldehyde in PBS for 20 min, and permeabilized with 0.05% Triton X-100 for 10 min. Actin was visualized using Alexa 647 conjugated Phalloidin (Invitrogen, Canada). Samples were mounted in Gold Anti-fade DAPI-containing mounting medium (Invitrogen, Canada) and visualized using an Olympus AX70 with a photometrics PXL cooled charge coupled device camera and Image-Pro Plus Software (Carsen Group, Ontario, Canada)

### Quantification of F-Actin Content by Flow Cytometry:

HASM cells were seeded in triplicate in 12-well plates, serum starved and stimulated with Sema3E (100 ng/ml) for 1-120 min. Cells were fixed, permeabilized and stained with Phalloidin as described earlier. Then, cells were detached by scrapers and intracellular fluorescence was determined using flow cytometry. After appropriate gating to remove cell debris from analysis, histograms of cell number versus log fluorescence intensity and forward angle light scatter were recorded for 10,000 HASM cells per sample. Fluorescence gain and photomultiplier voltage were identical for all samples. Relative F-actin content following Sema3E treatment was expressed as the mean fluorescence intensity (MFI) and was compared to that of non-stimulated HASM cells.

### Assessment of GTPase Activity:

For determination of membrane-anchored active vs. cytosolic inactive Rho and Rac GTPases, serum-deprived HASM cells were stimulated with Sema3E in the presence or absence of PDGF (10 ng/mL) for 0, 5 and 30 min. Cells were scraped in ice cold buffer (10 mM Tris-HCl, pH 7.5, 0.1 mM EDTA, 0.1 mM EGTA, 1 mM dithiothreitol, and protease inhibitor cocktail), sonicated on ice 3 times for 5 s, and then the homogenate was separated into cytoplasmic and membrane fractions by ultra-centrifugation (100,000 g for 45 min). The membrane fractions were solubilized in dissociation buffer (50 mM Tris-HCl, pH 7.5, 0.15 M NaCl, 1 mM dithiothreitol, 1% SDS, 1 mM EDTA, 1 mM EGTA, protease inhibitor cocktail), and subsequently size fractioned by 15% SDS-PAGE for immunoblot analysis using anti-Rac1/2/3 and anti-RhoA primary antibodies (Cell Signaling) (Ghavami, S., et al., Biochim Biophys Acta, 2010. 1803(4):452-67). As a membrane protein marker, Pan-cadherin abundance was used to normalize for loading of membrane fractions.

### Statistical Analysis:

Values were presented as the means±SEMs of at least three independent experiments. The statistical differences between pairs were determined by Mann-Whitney U test. Differences were considered to be statistically significant at P<0.05.

### Results

### Sema3E and its Holoreceptor Components are Expressed in HASM Cells.

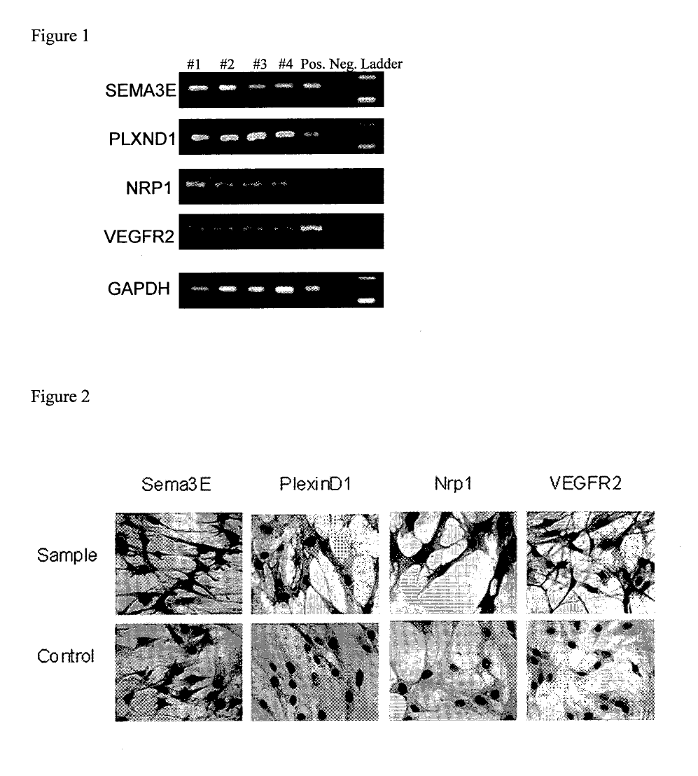

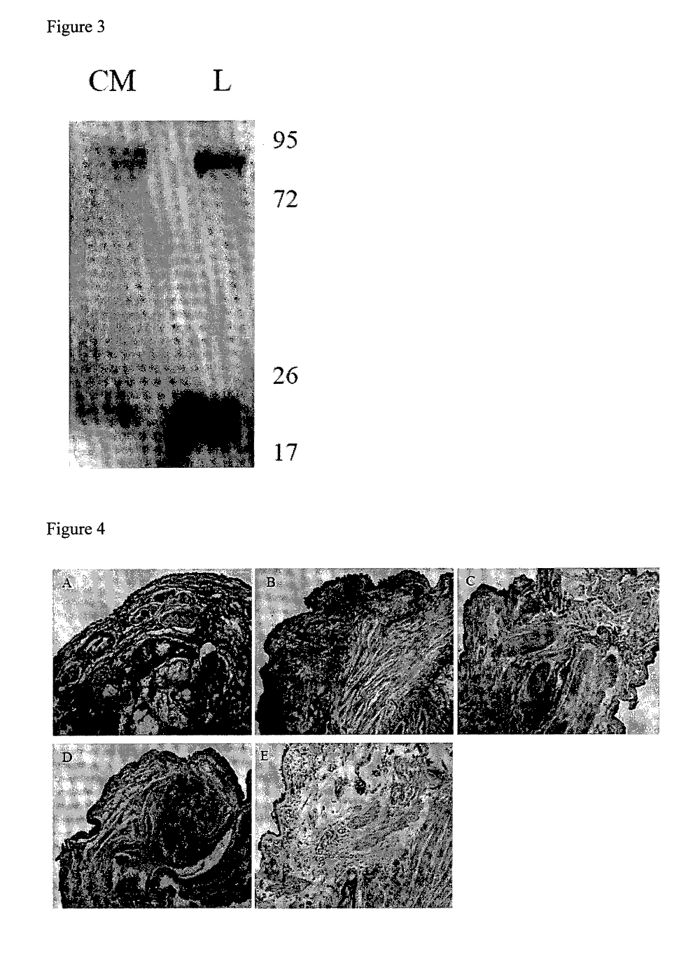

It has been previously shown that Sema3E is expressed in neuronal and cardiovascular systems and directly binds to PlexinD1 as a receptor. However, depending on biological circumstances, Sema3E can also be gated by Nrp1 or VEGFR2 as (co)receptors. In vitro expression of Sema3E and its potential receptors in HASM cells was evaluated at mRNA and protein level with RT-PCR and immunocytochemistry respectively. As shown in FIG. 1, mRNA for SEMA3E, PLXND1, NRP1 and VEGFR2 was expressed in four different HASM cells. In parallel, our immunocytochemistry experiments using specific antibodies demonstrated their basal protein expression, as well (FIG. 2). Production and secretion of Sema3E protein was further studied by performing Western blot analysis on both HASM cell lysate and conditioned medium and in both of them Sema3E was detected in two isoforms: full-length p 87.5 and p25 kDa fragments (FIG. 3). Collectively, this part of our studies revealed that Sema3E and receptors are stably expressed in HASM cells.

### Sema3E is Differentially Expressed in Asthmatic Airways Compared to Non-Asthmatic Subjects.

Expression of Sema3E might be correlated with the progression of allergic asthma. To investigate expression pattern of Sema3E in vivo, we performed immunohistochemical analysis using human bronchial biopsy specimens. As shown in FIG. 4A, Sema3E is highly expressed in normal HASM cells compared to asthmatic subjects (FIG. 4B-D) where Sema3E expression is repressed in HASM cells in parallel to disease severity. In fact, our data revealed that Sema3E is inversely correlated with asthma progression. Negative isotype controls using rabbit IgG staining showed no immunoreactivity in specimens (FIG. 4E).

### Basal and Growth Factor-Induced HASM Cell Migration and Proliferation is Inhibited by Sema3E in a Dose Dependent Manner.

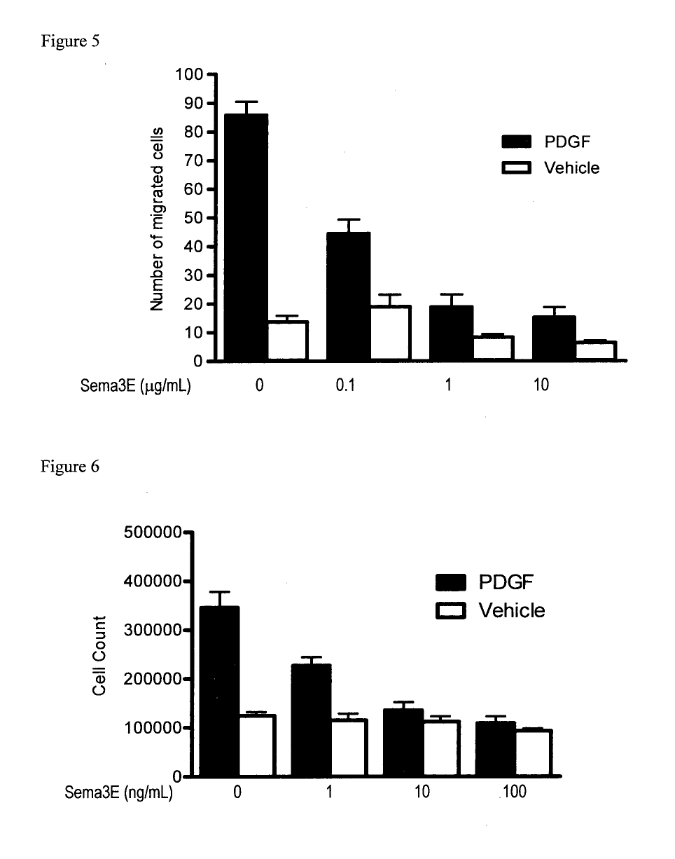

To evaluate HASM cell migration in response to Sema3E, a Boyden chamber assay specifically developed to study smooth muscle cell migration was used as described previously (Goncharova, E. A., et al., Nat Protoc, 2006. 1(6):2933-9). Cultures were treated with Sema3E±PDGF and migrated cells were counted 4 hours later. FIG. 5 reveals the significant inhibitory effect of Sema3E on both basal and PDGF-induced HASM cell migration in a dose dependent manner.

To study effects of Sema3E on proliferation, HASM cells stimulated with Sema3E±PDGF for four days were collected and counted with a hemocytometer. As shown in FIG. 6, basal and PDGF-induced proliferation of HASM cells was inhibited by Sema3E dose-dependently with a significant decrease in cell number.

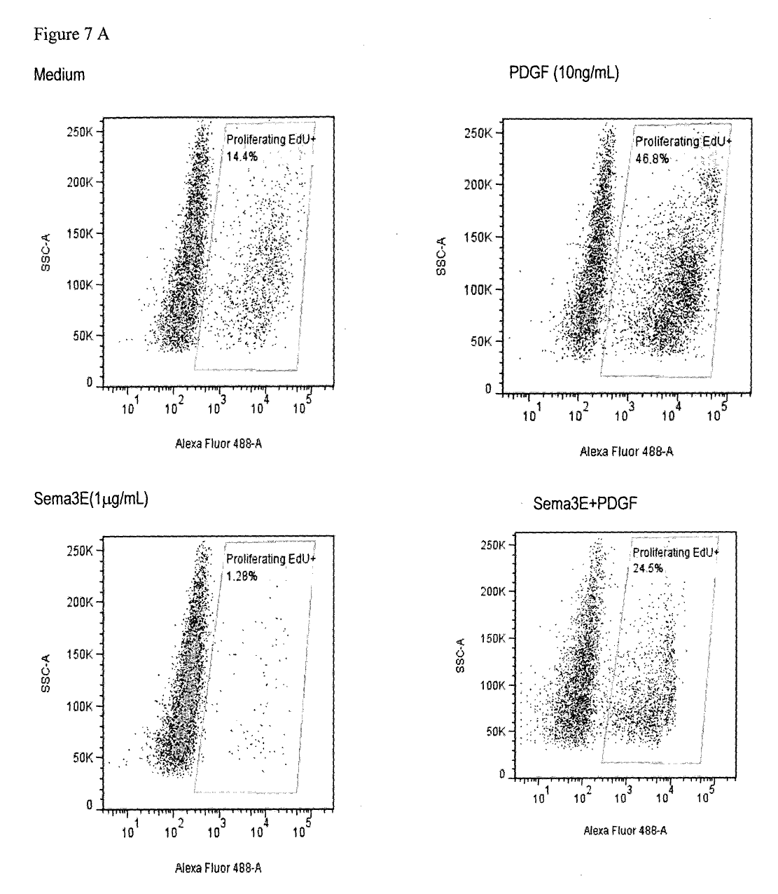

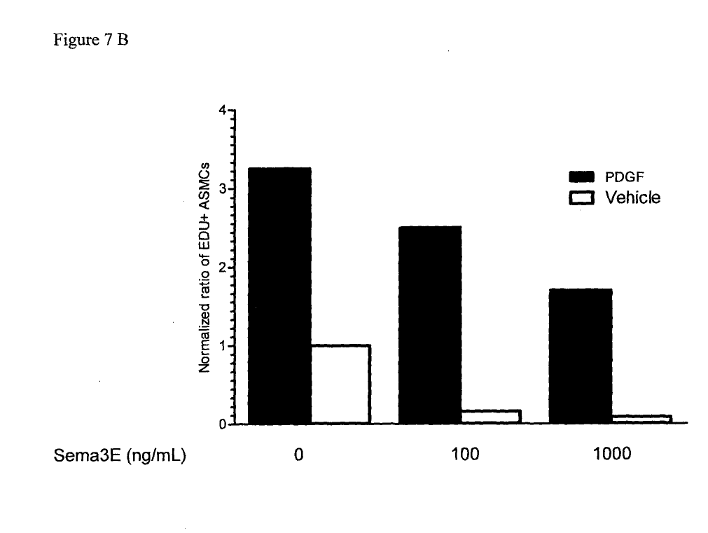

In a different set of experiments, effect of Sema3E on HASM cell proliferation was investigated by measuring incorporated EdU into newly synthesized DNA following Sema3E±PDGF treatments. Results of EdU proliferation assay confirmed inhibitory effect of Sema3E on HASM cell proliferation (FIG. 7).

Collectively, our data indicates Sema3E significantly affects essential aspects of HASM cell function in terms of proliferation and migration.

### Actin Depolymerization is Induced Following Sema3E Stimulation in HASM Cells.

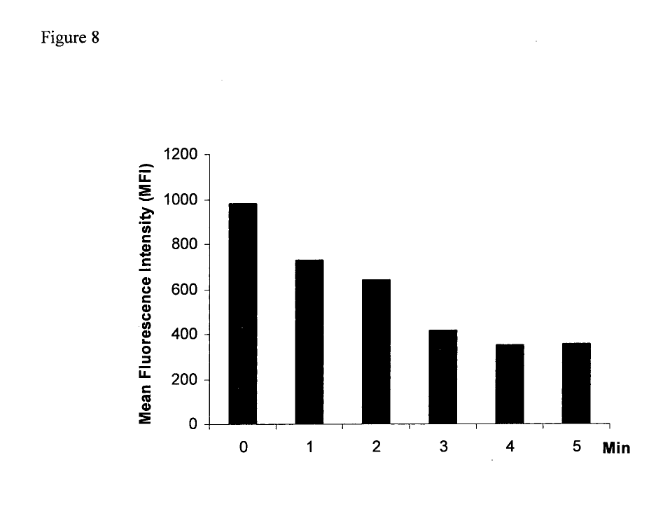

Alexa-647-conjugated Phalloidin was used to study actin alterations after Sema3E treatment of HASM cells and results were quantified as MFI values. Our data shows a significant rapid decrease in F-actin content of HASM cells in response to Sema3E (FIG. 8).

### Discussion

### Findings:

In this study, we have revealed for the first time that a member of semaphorin family (Sema3E) and its receptors are expressed in HASM cells and affect HASM cell proliferation and migration. We also showed that Sema3E expression in human airways (ASM bundle) is decreased in accordance to asthma progression. Moreover, we have shown that effect of Sema3E on HASM cells is associated with actin rearrangement.

### Importance of HASM Cell Proliferation and Migration.

Several lines of evidence support that HASM cell proliferation and migration contribute to observed smooth muscle hyperplasia in asthmatic airways (Johnson, P. R., et al., Am J Respir Crit Care Med, 2001. 164(3):474-7, Gerthoffer, Proc Am Thorac Soc, 2008. 5(1):97-105). Therefore, inhibiting HASM cell proliferation and migration has been proposed as a rational strategy to develop new anti-asthmatic drugs (Bai, T. R., Curr Opin Allergy Clin Immunol, 2010. 10(1):82-6, Damera, G., et al., Pulm Pharmacol Ther, 2009. 22(5):353-9) as the mechanism of action for current therapeutics might be to some extent through inhibition of HASM cell proliferation and migration. However, regulatory mechanisms involved in these complex processes have remained to be clearly defined. Deciphering endogenous mediators triggering signaling pathways which lead to modulation of HASM cell functions is an essential step towards understanding pathogenesis of asthma and development of novel therapeutic strategies.

### Importance of Semaphorins.

Playing multifaceted roles in various circumstances, semaphorins have been shown to be involved in pathogenesis of several diseases from different types of cancer to autoimmune and neurological disorders (Gaur, P., et al., Clin Cancer Res, 2009. 15(22):6763-70, Makino, N., et al., FEBS Lett, 2008. 582(28):3935-40, Mann, F., et al., Prog Neurobiol, 2007. 82(2):57-79, Neufeld and Kessler, Nat Rev Cancer, 2008. 8(8):632-45, Vadasz, Z., et al., Autoimmun Rev, 2010. 9(12):825-9). In addition, it has been shown that Sema3A expression is decreased in atopic dermatitis and more interestingly its alleviative efficacy has been demonstrated in an animal model of this allergic disease (Tominaga, et al., Br J Dermatol, 2008. 158(4):842-4, Yamaguchi, J., et al., J Invest Dermatol, 2008. 128(12):2842-9).

### Sema3E Expression.

Here, we have demonstrated that HASM cells express and secrete Sema3E in two different isoforms: p25 and p87.5 kDa. It was previously shown that enzymatic cleavage of Sema3E leads to generation of a p61 kDa fragment from full-length p87.5 kDa which converts repelling anti-migratory effect of Sema3E to attracting pro-migratory one (Christensen, C., et al., Cancer Res, 2005. 65(14):6167-77). The p61 isoform is predominantly produced in metastatic cancer cells and is involved in tumor progression (Christensen, C., et al., Cancer Res, 2005. 65(14):6167-77, Casazza, A., et al., J Clin Invest, 2010. 120(8):2684-98). But, in our study, p61 fragment was not desirably detected in cultured HASM cells neither in cell lysate and conditioned medium.

### Receptors.

We have also shown expression of all plausible Sema3E receptor components including PlexinD1, Nrp1 and VEGFR2. It should be noted that unlike other class 3 semaphorins, Sema3E interacts directly with Plexin D1, but does not bind Nrp1 in endothelial cells and exerts its repulsive roles during vasculature independently of Nrp1 as a co-receptor (Gu, C., et al., Science, 2005. 307(5707):265-8). However, gating of Sema3E-PlexinD1 complex by Nrp1 switches repulsive signals to attractive ones (Chauvet, S., et al., Neuron, 2007. 56(5):807-22). More recently, VEGFR2 has been identified as an additional obligatory component to induce Sema3E attractive signals (Bellon, A., et al., Neuron, 2010. 66(2):205-19). Nrp1 and VEGFR2 both function as Sema3E co-receptors during brain development and despite their expression in normal HASM cells; it seems that Sema3E signaling in these cells leading to anti-proliferative and anti-migratory responses is exclusively mediated by PlexinD1 and is independent of co-receptors. On the other hand, it has been shown that VEGFR2 mRNA expression is significantly increased in asthmatic tracheal tissues compare to normal subjects (Su, X., et al., Pathobiology, 2008. 75(1):42-56).

Expression of both ligand and receptor on HASM cells indicates probable autocrine manner of Sem3E-PlexinD1 signaling in these cells. Furthermore, PlexinD1 is expressed in endothelial cells and it also proposes a potential paracrine way of signaling leading to modulate angiogenesis that is augmented in asthmatic airways as a pathological phenomenon. Angiogenesis may result from endothelial cell proliferation and migration, recruitment of perivascular supporting cells, and a maturation process (Bischof, R. J., et al., Proc Am Thorac Soc, 2009. 6(8):673-7). It has been demonstrated that VEGF as the master mediator of angiogenesis enhances the T helper 2 (Th2) response as well as ASM hyperplasia (Wilson, et al., Curr Opin Allergy Clin Immunol, 2006. 6(1):51-5). Th2 cytokines induce structural cells such as ASM cell to produce VEGF, which in turn, increase allergen-induced inflammation and consequent remodeling (Puxeddu, et al., J Allergy Clin Immunol, 2005. 116(3):531-6). Emerging studies have revealed anti-angiogenic effect of Sema3E on VEGF-treated endothelial cells (Choi, Y. I., et al., Immunity, 2008. 29(6):888-98, Moriya, J., et al., Circ Res, 2010. 106(2):391-8) through rapid disassembly of integrin-mediated adhesive structures leading to inhibition of endothelial cell adhesion to the extracellular matrix.

### Function (Proliferation Migration).

Using different functional approaches, our data indicates that Sema3E inhibits essential aspects of HASM cell function including proliferation and migration. From a functional point of view, semaphorins are more likely to provoke repulsion of target cells than attraction. For instance, Sema3A and Sema4D induce axonal collapse in growth cone region of neuronal cells (Fuchikawa, et al., Biochem Biophys Res Commun, 2009. 385(1):6-10, Mikule, et al., J Neurosci, 2002. 22(12):4932-41). Sema3F also inhibits endothelial and tumor cell migration and adhesion (Bielenberg, et al., Methods Enzymol, 2008. 443:299-314). Conversely, Sema5A is able to promote angiogenesis by increasing endothelial cell proliferation, migration, and decreasing apoptosis (Sadanandam, et al., Microvasc Res, 2010. 79(1):1-9). Even an individual semaphorin can induce either repulsive or attractive responses and the functional outcome (repulsion vs. attraction) depends on encountered biological milieu in a cell-type-dependent manner (Zhou, et al., Trends Biochem Sci, 2008. 33(4):161-70). As a bifunctional protein, Sema3E effect on target cells is determined by proteolytic processing and interaction with co-receptors. Regarding importance of HASM cell proliferation and migration in airway remodeling, the observed inhibitory effect of Sema3E on these processes suggests us to assess Sema3E functions in animal model of asthma. Besides Sema3E probable roles in structural changes namely airway remodeling, it might also reduce airway inflammatory responses; as it has been reported that Sema3A can block IL-23 production and IL-6 and TNF-α secretion in peripheral blood mononuclear cells derived from individuals with active rheumatoid arthritis that eventually leads to reduce progression of the disease in an experimental model (Catalano, J Immunol, 2010. 185(10):6373-83).

### Signaling.

In an effort to understand the mechanism of Sema3E action on HASM cell proliferation and migration, a dramatic decrease in polymerized form of actin (F-actin) rapidly following Sema3E stimulation was observed. Semaphorin signaling through receptors is mainly converged to cytoskeleton through regulation of actin rearrangement or modulation of integrin-mediated cell adhesion (Capparuccia and Tamagnone, J Cell Sci, 2009. 122(Pt 11):1723-36). Recent findings have revealed that a family of flavoprotein monooxygenases called “molecule interacting with Cas ligand” (MICAL) is both necessary and sufficient for Semaphorin-Plexin mediated F-actin reorganization in neurons (Hung, R. J., et al., Nature, 2010. 463:823-7), but it has not yet been studied in the other cells. If Semaphorin-Plexin-MICAL axis proves to be important in contexts outside the nervous system such as airways, it could become a relevant target for innovative anti-asthmatic drugs.

Two overall mechanisms of Semaphorin-Plexin signaling include regulation of actin by as homology (Rho) proteins (e.g. Ras-related C3 botulinum toxin substrate 1 or Rac-1) or modulation of integrin-mediated cell adhesion via “Rat Sarcoma” (Ras) molecules (e.g. R-Ras). These monomeric p21 GTP-binding proteins (small GTPases) are early signaling components that regulate cellular functions through hydrolysis of GTP and cycling from the GDP to the GTP-bound state. When bound to GTP, small GTPases are activated and attach to effector proteins to carry out a cascade of events including cell migration, proliferation, and angiogenesis (Thou, et al., Trends Biochem Sci, 2008. 33(4):161-70), (Capparuccia and Tamagnone, J Cell Sci, 2009. 122(Pt 11):1723-36), and (Puschel, Adv Exp Med Biol, 2007. 600:12-23). On the other hand, it has been shown that smooth muscle cell proliferation and migration (as well as angiogenesis) are also mediated by small GTPases (Simeone-Penney, M. C., et al., Am J Physiol Lung Cell Mol Physiol, 2008.294(4):L698-704, Page, K., et al., J Biol Chem, 1999. 274(31):22065-71, Spindler, et al., Cardiovasc Res, 2010. 87(2):243-53, Sakata, et al., Arterioscler Thromb Vasc Biol, 2004. 24(11):2069-74).

ASM cells are dynamically involved in several aspects of airway biology (pathology) because of their responsiveness to various stimulations in terms of proliferation, migration, contraction, cytokine production and synthesis of extracellular matrix components (Halayko, et al., Curr Drug Targets, 2006. 7(5):525-40, Hirst, Respir Physiol Neurobiol, 2003. 137(2-3):309-26). Therefore, modulation of ASM cell function has emerged as a potential therapeutic strategy to treat asthma. Even β₂-adrenergic receptor agonists and glucocorticoids as current asthma drugs regulate ASM cell proliferation and migration negatively Goncharova, E. A., et al., Am J Respir Cell Mol Biol, 2003. 29(1):19-27, Kassel, K. M., et al., Am J Physiol Lung Cell Mol Physiol, 2008. 294(1):L131-8, Stewart, A. G., et al., Mol Pharmacol, 1999. 56(5):1079-86, Stewart, A. G., et al., Br J Pharmacol, 1997. 121(3):361-8). Regarding efficacy and safety issues in development of novel therapeutic strategies, deciphering endogenous mediators able to inhibit pathological functions of ASM cells could be an urgent priority. As a novel approach, our study provides the concept of semaphorin contribution in ASM cell function and airway remodeling.

### Conclusion

In conclusion, we demonstrate for the first time the expression of a semaphorin in HASM cells and its inhibitory effect on basal and growth factor-induced HASM cell proliferation and migration. Furthermore, we find in vivo differential expression pattern of Sem3E in (smooth muscle bundles of) asthmatic vs. healthy airways. We also show induction of actin reorganization (and association of small GTPases as early signaling mediators) in response to Sema3E. Collectively, these results provide a new mechanism through which Sema3E inhibits HASM cell proliferation and migration providing a new clue to control airway remodeling and clinical manifestations of asthma.

### Example 2

### Materials and methods

### Mice:

Female BALB/c and C57BL/6 mice (18-20 grams, 6-week-old) were obtained from the Central Animal Care Services, University of Manitoba, housed in university animal facilities and maintained according to the recommendations of the Canadian Council of Animal Care. All experimental protocols were approved by the Animal Care and Use Committee at the University of Manitoba.

### HDM-Induced Acute Asthma:

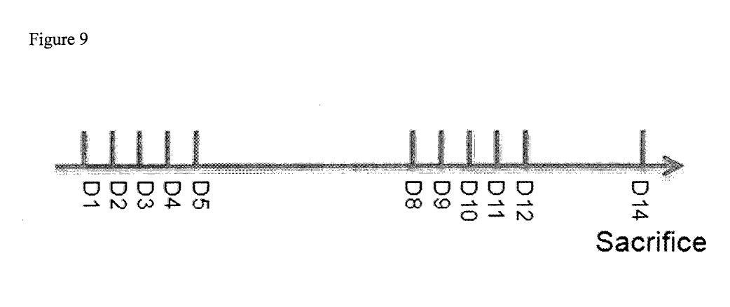

Lyophilized house dust mite or HDM (Dermatophagoides pteronyssinus) extract was purchased from Greer Laboratories, Inc. (Lenoir, N.C.) and reconstituted in sterile endotoxin-free saline as 2.5 mg/mL stock concentration before treatment. Working concentration (25 μg per mouse in 35 μL of saline) was freshly prepared and acute asthma was induced by intranasal inhalation of HDM, for 5 days per week for 2 consecutive weeks (FIG. 9). Recombinant mouse Sema3E-Fc (10 μg/kg in sterile PBS) was intra-nasally administered 1 hour prior to HDM challenge. Control group (vehicle-treated) received sterile LPS free saline at the same time points. All experiments were performed 48 hours after the last HDM exposure.

### Airway Hyperreactivity (AHR Measurement):

Following anesthesia with intra-peritoneal injection of pentobarbital (0.1 mL per 10 grams body weight), mid-cervical tracheotomy and tracheal cannulation was performed using a polyethylene catheter (1.1 25 mm). The catheter was coupled to a FlexiVent small animal ventilator (Scireq, Montreal, Quebec) and positive end expiratory pressure was maintained at 3 cmH₂O. The ventilator delivered a tidal volume of 10 mL air per kg body weight at a rate of 150 breaths/min. Then, mice were subjected to serial aerosol metacholine (MCh) challenge (0, 3, 6, 12, 25, and 50 mg/mL in 30 μL of saline), and baseline mechanics were determined using saline-only challenge. Before each challenge with saline or MCh, lung loading history was normalized by inflation to total lung capacity. Respiratory mechanics were assessed using a preset FlexiVent Prime-8 low-frequency forced oscillation protocol to derive respiratory mechanical input impedance (Zrs). Airway resistance (R_{aw}), tissue resistance (G) and lung elastance (H) were derived by fitting Zrs to the constant phase model.

### Bronchoalveolar Lavage:

Bronchoalveolar lavage (BAL) was performed using 2 instillations of 1 mL sterile saline. BAL fluid was centrifuged at 1500 rpm/4° C. for 10 min and supernatant was separated and stored at −80° C. to measure cytokines. Pellet was gently resuspended in 1 mL sterile saline and total cells were counted using a hemocytometer.

### Cytology:

Based on total BAL cell count results, 10⁵-1.5×10⁵ cells were spun (1500 rpm/5 min) onto the slides. Cytospins were dried overnight at room temperature and then frozen at −20° C. until staining. After staining, cells were characterized morphologically and 200 cells/slide were differentially counted using 400× magnification by two individuals in a double-blind manner.

### BAL Flow Cytometry:

Following RBC lysis with ACK buffer and Fc blocking for 2 and 15 min respectively, BAL cells were resuspended in 0.5 mL of complete DMEM. Cells were washed by flow buffer and stained by appropriate fluorochrome-conjugated antibodies to detect extracelluar surface markers as described by Rijt, et al. (JIM, 2004, 288: 111-121). The following monoclonal Abs were added to the cells and incubated for 30 minutes on ice away from light: MHCII-APC, CD11c-FITC, CD3-Cy7 (BD Bioscences, San Diego, Calif.) and CCR3-PE (R&D systems Inc., Minneapolis, Minn.). Appropriate compensation control for each Ab was also included in the experiment. After fixation using 2% paraformaldehyde (15 minutes, on ice and away from light), samples were extensively washed by flow buffer 3 times, acquired by BD FACSCanto II flow cytometer and analyzed using FlowJo software.

Lymphocytes had FSC^{lo})/SSC^{lo} scattering pattern and B cells as antigen presenting cells were discriminated from T cells by MHCII expression in the CD3⁺ gate. Granulocytes were recognized as nonautofluorescent highly granular (SSC^{hi}) cells including eosinophils as CCR3⁺ CD3^{lo/-} and MHCII^{lo/-} cells and also neutrophils as SSC^{hi} but CCR3⁻ cells. Dendritic cells had CD3⁻/MHCII^{hi} and CD11c^{hi} phenotype which were differentiated from large autofluorescent alveolar macrophages expressing intermediate levels of MHCII and CD11c.

### Results

### Sema3E Inhibits HDM-Induced AHR in Mice:

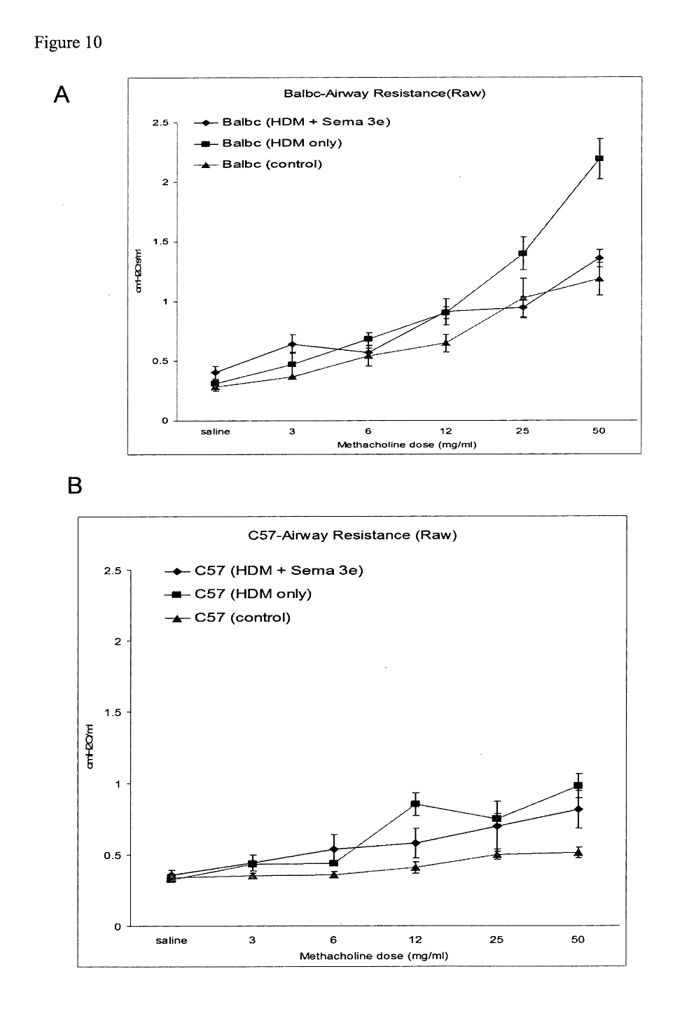

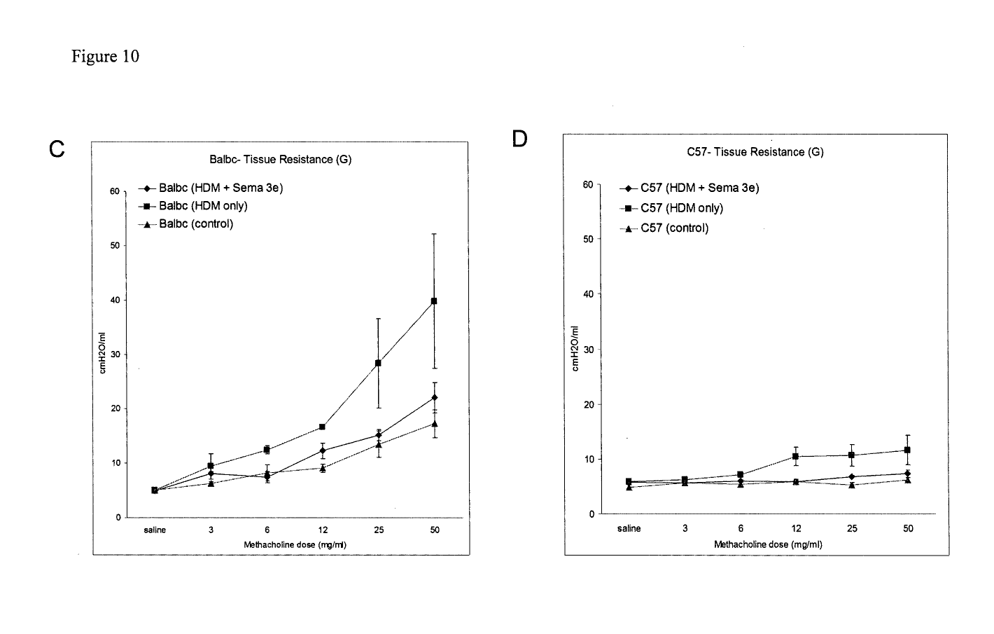

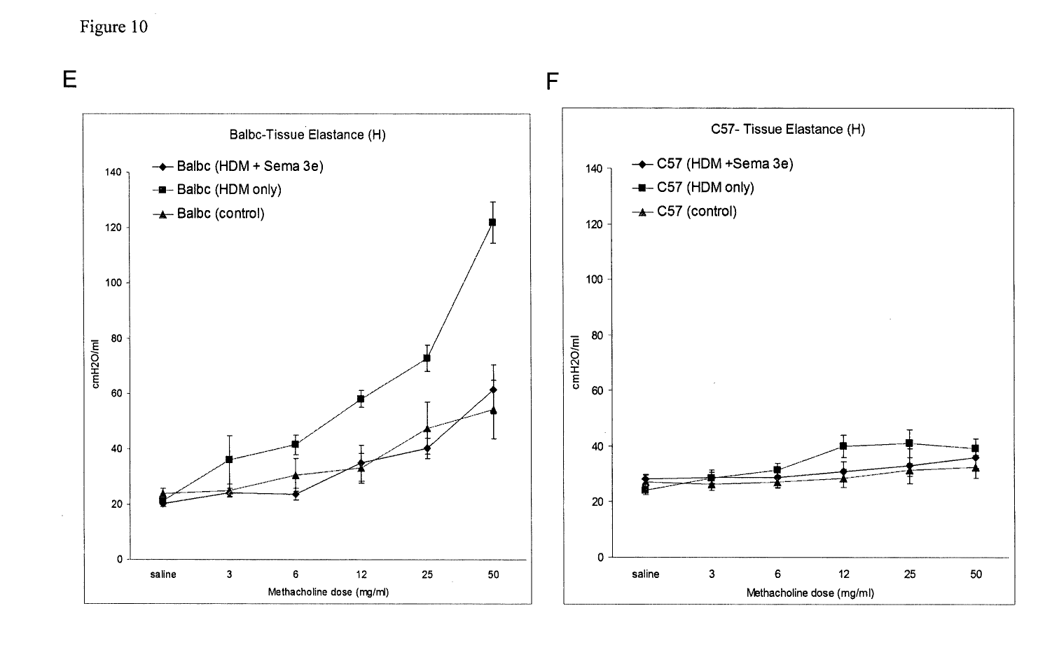

To determine whether Sema3E treatment during allergen challenge could influence the development of airway hyper-reactivity, Sema3E was administered intra-nasally to the mice 1 hour before each HDM challenge. HDM-challenged mice, as a positive control group, showed a dramatic increase AHR parameters, including airway resistance (R_{aw})(FIG. 10A-B), tissue resistance (G)(FIG. 10C-D) and lung elastance (H) (FIG. 10E-F), in response to various doses of aerosolized MCh. Intra-nasal application of Sema3E prior to allergen challenge led to significant attenuation of AHR parameters in both BALB/c and C57BL/6 mice (FIG. 10). Basal AHR to MCh in saline-treated (naïve) mice was also studied as a negative control group.

### Sema3E Reduces HDM-Induced Airway Inflammation in Mice:

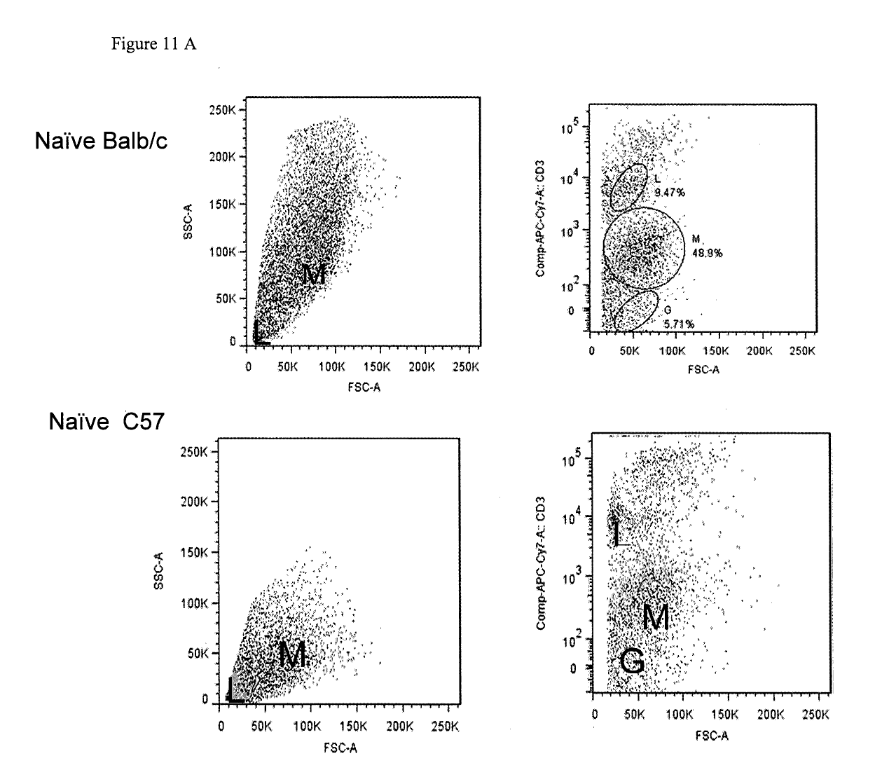

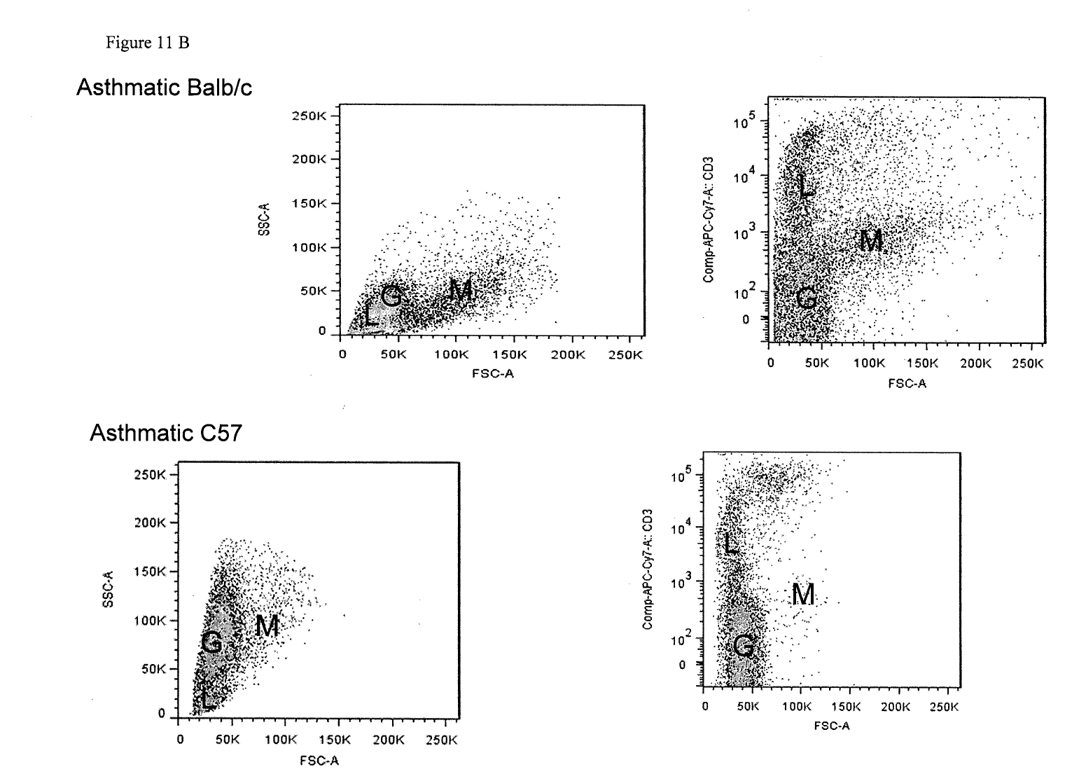

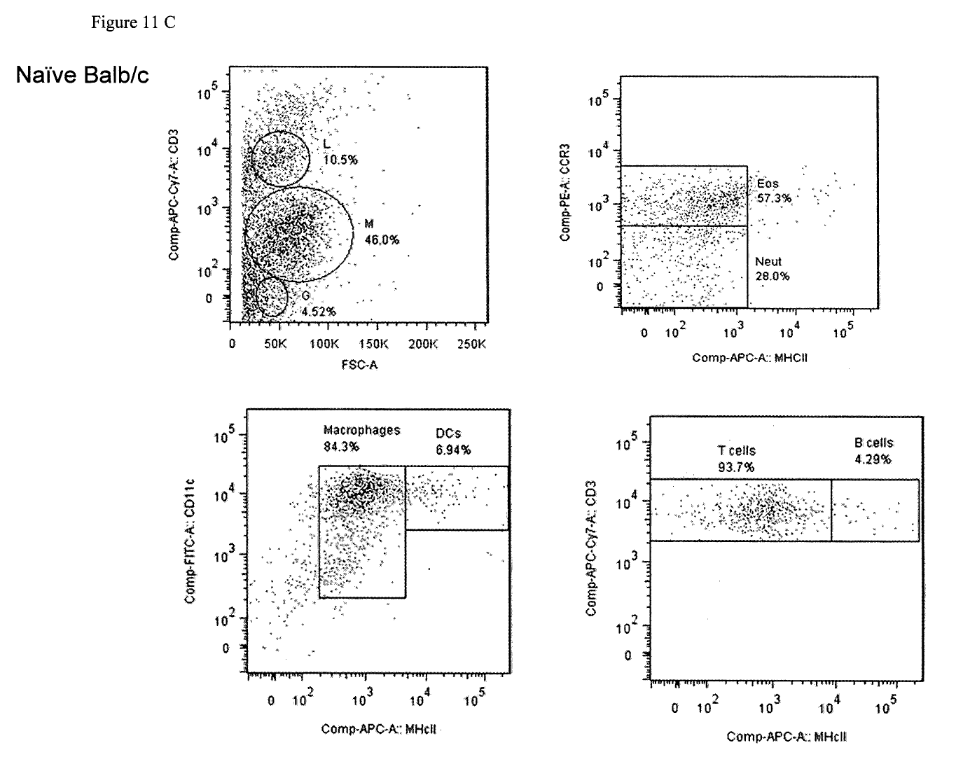

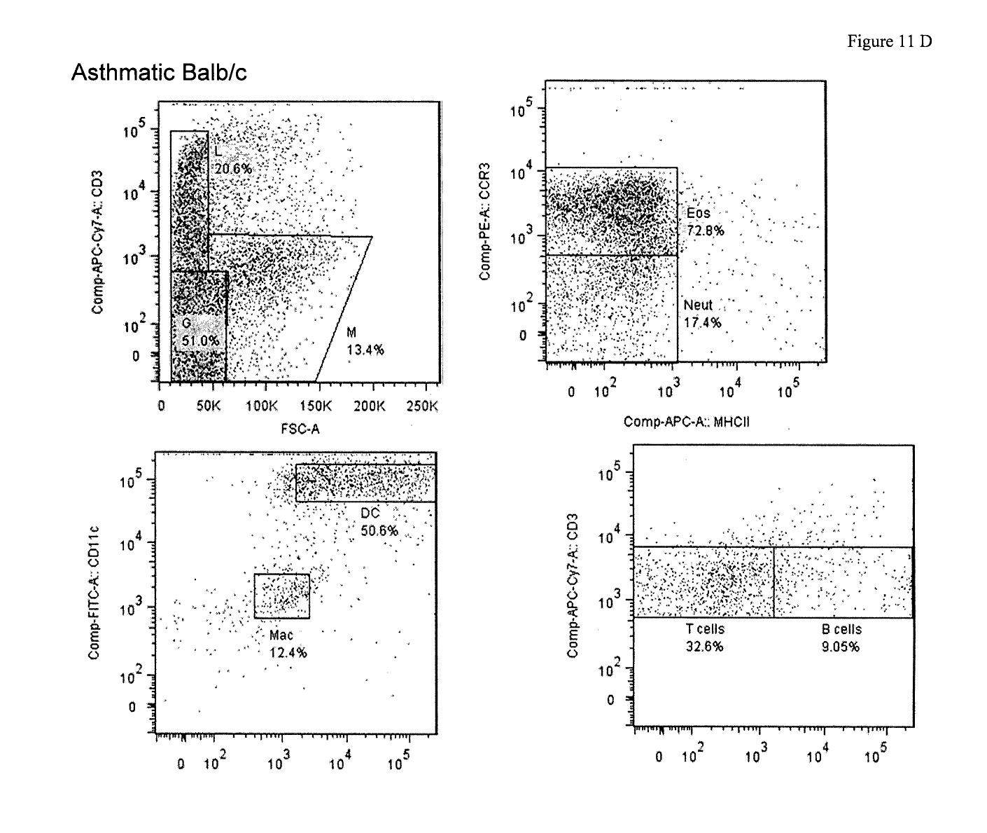

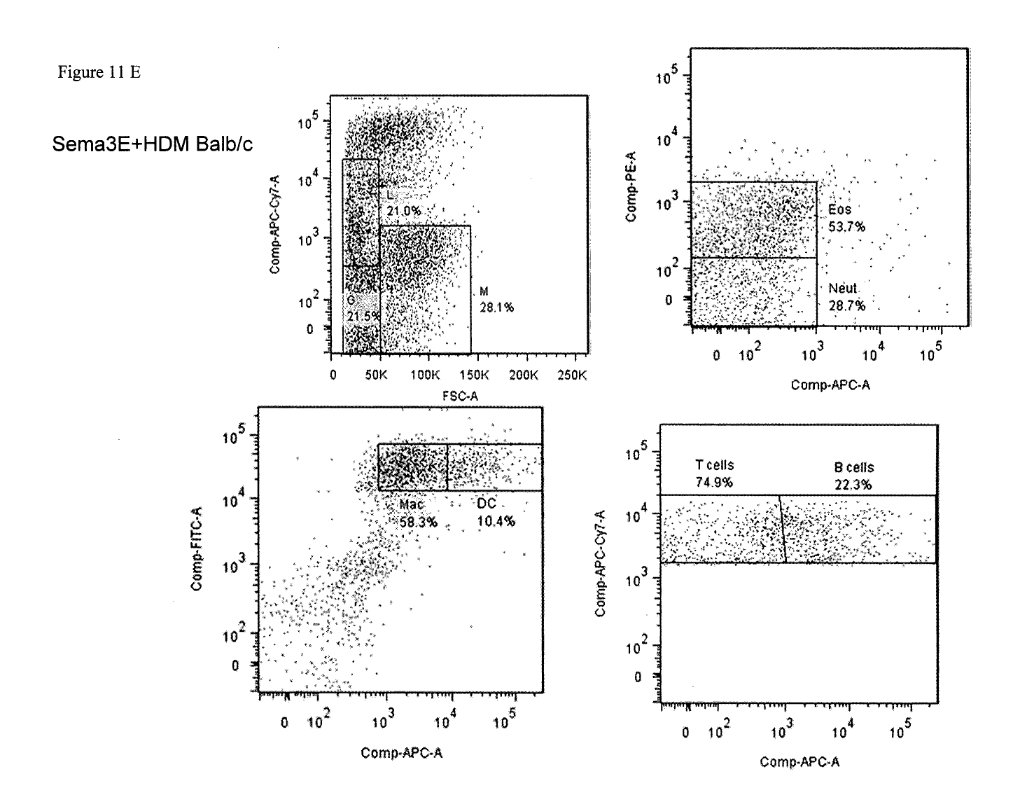

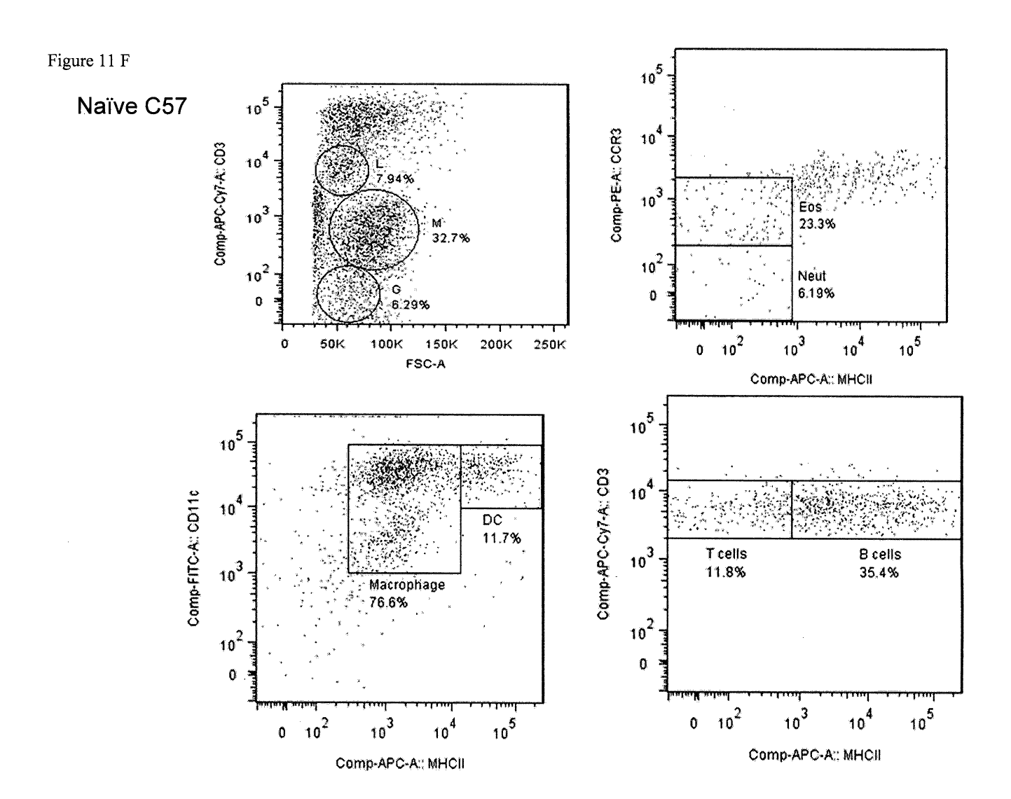

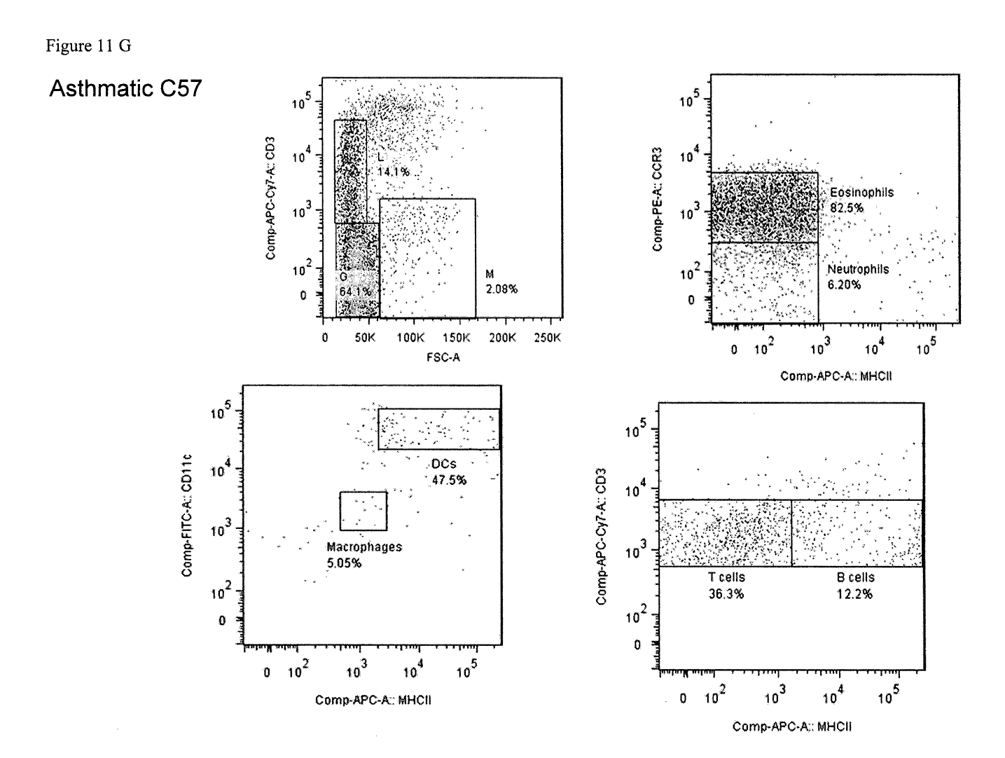

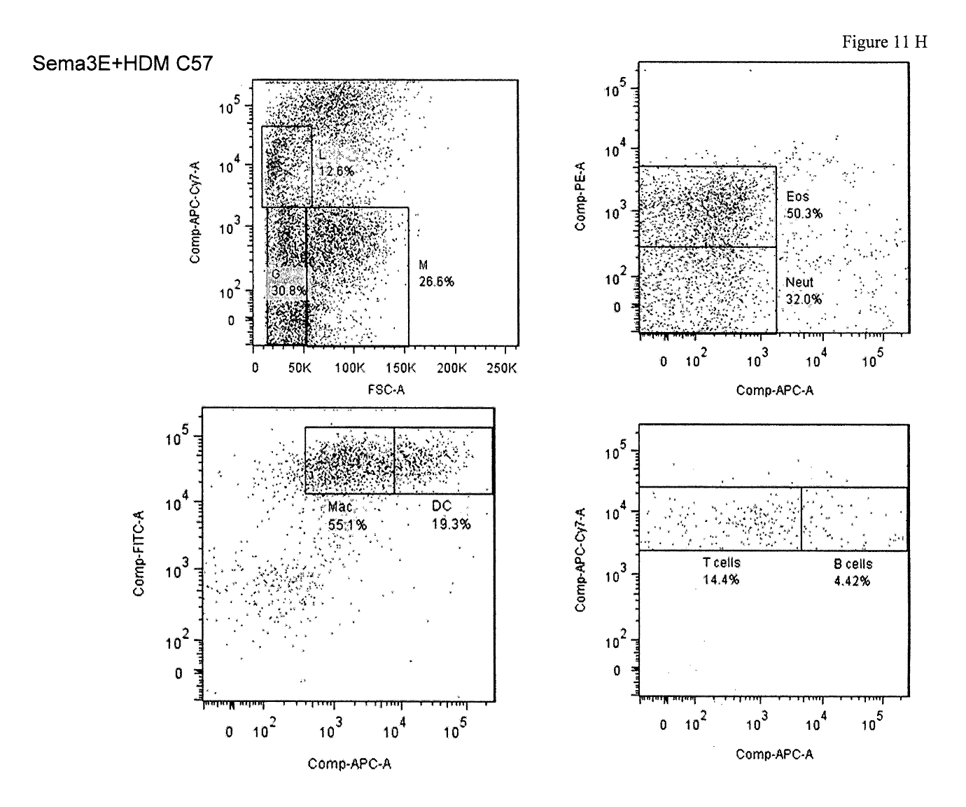

In order to further investigate the effects of Sema3E on acute airway allergies, BALF samples were collected from the all aforementioned mice groups that had undergone tracheotomy and MIR measurement. Total BALF cells as well as differential inflammatory cell count was performed by routine cytological methods and further characterized using flow cytometry. As seen in FIG. 11D, BALF eosinophilia as a cardinal feature of allergic airway inflammation was tremendous in HDM-challenged mice. Surprisingly, intra-nasal treatment of Sema3E prior to HDM exposure resulted in significant reduced BALF eosinophilia (FIG. 11E) as well as restored mononuclear cell population almost comparable to naïve (saline treated) mice (FIG. 11F).

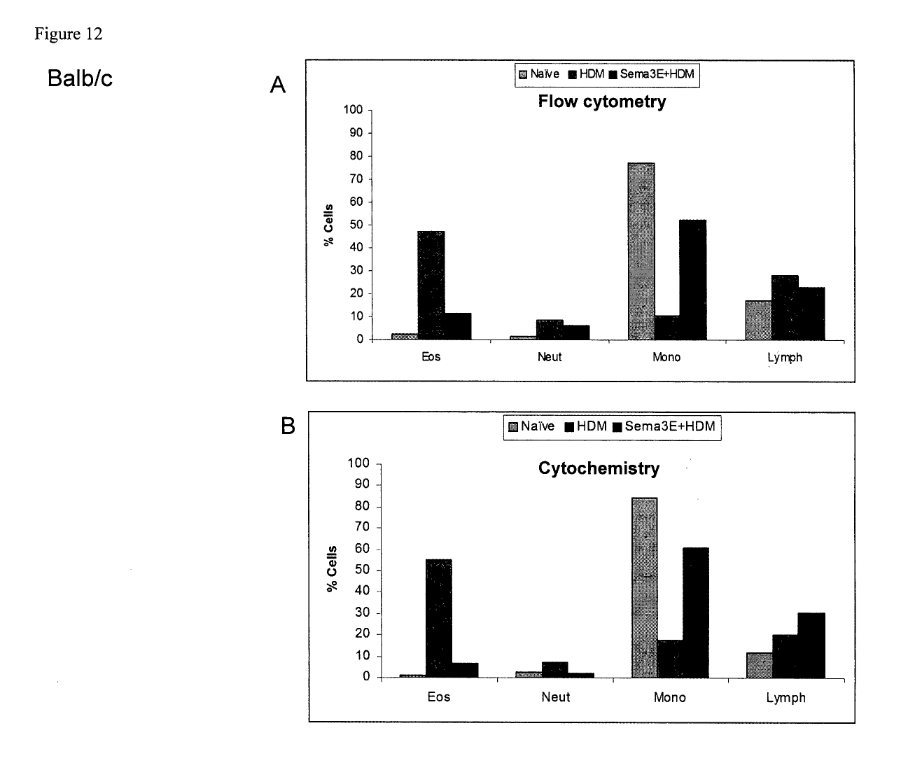

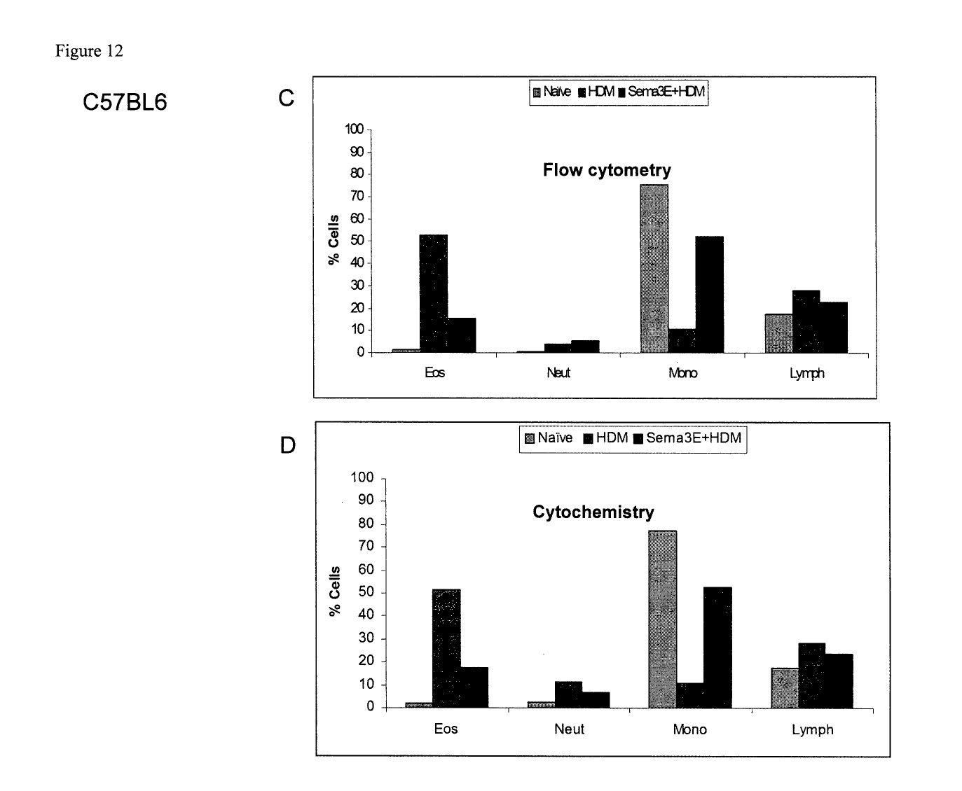

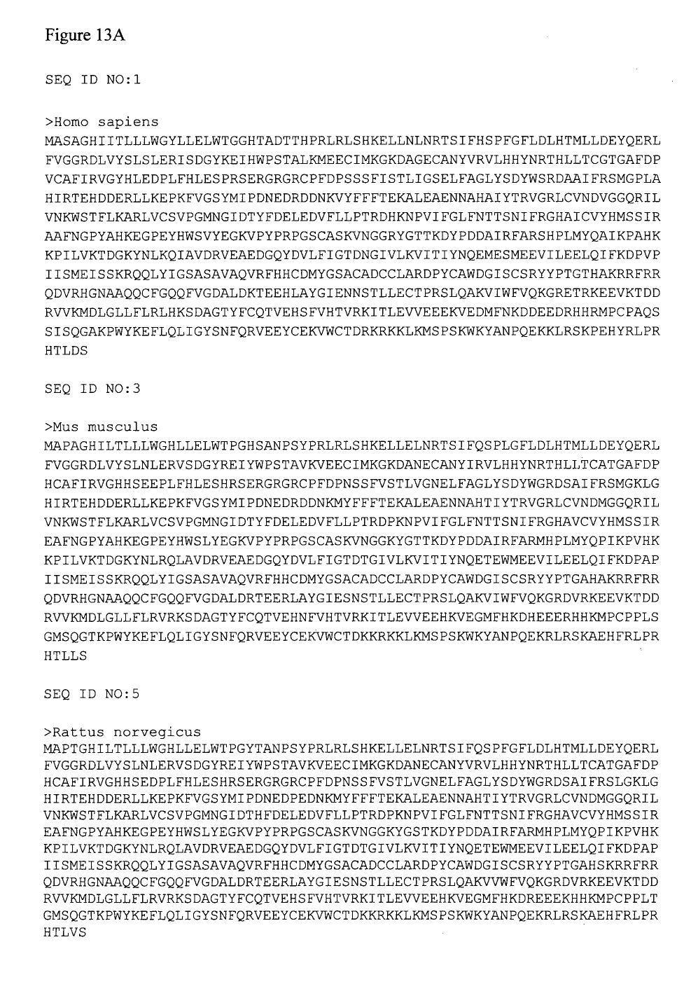

These results were also observed in C57BL6 strain (FIG. 3G-H) and similar results were obtained using cytology and flow cytometry (FIG. 12).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. Use of a sema3E polypeptide and a pharmaceutically acceptable carrier, in the preparation of a medicament for an inflammatory airway disease.

2. Use of a sema3E polypeptide and a pharmaceutically acceptable carrier, for treating an inflammatory airway disease.

3. The use of claim 1 or 2 wherein the sema3E polypeptide comprises a KRRXRR consensus site, wherein X is any amino acid.

4. The use of claim 3 wherein the sema3E polypeptide further comprises an RXXR consensus site, wherein X is any amino acid.

5. The use of claim 4 wherein the sema3E polypeptide further comprises a second RXXR consensus site, wherein X is any amino acid.

6. The use of claim 1 or 2 wherein the sema3E polypeptide comprises a Sema domain.

7. The use of claim 6 wherein the sema3E polypeptide further comprises one or more domains selected from a cystine rich domain, an immunoglobulin domain, and a short basic domain.

8. The use of claim 6 wherein the sema3E polypeptide further comprises a cystine rich domain and an immunoglobulin domain.

9. The use of claim 1 or 2 wherein the sema3E polypeptide comprises an amino acid sequence having at least 80% identity with SEQ ID NO:2.

10. The use of claim 1 or 2 wherein the inflammatory airway disease is selected from acute asthma and chronic asthma.

11. The use of claim 1 or 2 wherein the sema3E polypeptide is a fusion polypeptide.

12. The use of claim 1 or 2 wherein the sema3E polypeptide has activity when determined by ability to bind to Plexin D1, ability to inhibit airway smooth muscle cell migration or proliferation, or ability to influence development of airway hyper-reactivity during allergen challenge.

13. Use of a sema3E polynucleotide and a pharmaceutically acceptable carrier, in the preparation of a medicament for an inflammatory airway disease, wherein the sema3E polynucleotide encodes a sema3E polypeptide.

14. Use of a sema3E polynucleotide and a pharmaceutically acceptable carrier, for treating an inflammatory airway disease, wherein the sema3E polynucleotide encodes a sema3E polypeptide.

15. The use of claim 13 or 14 wherein the sema3E polypeptide comprises a KRRXRR consensus site, wherein X is any amino acid.

16. The use of claim 15 wherein the sema3E polypeptide further comprises an RXXR consensus site, wherein X is any amino acid.

17. The use of claim 16 wherein the sema3E polypeptide further comprises a second RXXR consensus site, wherein X is any amino acid.

18. The use of claim 13 or 14 wherein the sema3E polypeptide comprises a Sema domain.

19. The use of claim 18 wherein the sema3E polypeptide further comprises one or more domains selected from a cystine rich domain, an immunoglobulin domain, and a short basic domain.

20. The use of claim 18 wherein the sema3E polypeptide further comprises a cystine rich domain and an immunoglobulin domain.

21. The use of claim 13 or 14 wherein the sema3E polypeptide comprises an amino acid sequence having at least 80% identity with SEQ ID NO:2.

22. The use of claim 13 or 14 wherein the inflammatory airway disease is selected from acute asthma and chronic asthma.

23. The use of claim 13 or 14 wherein the sema3E polypeptide is a fusion polypeptide.

24. The use of claim 13 or 14 wherein the sema3E polynucleotide is present in a vector.

25. The use of claim 24 wherein the vector is a viral vector.

26. The use of claim 13 or 14 wherein the sema3E polypeptide has activity when determined by ability to bind to Plexin D1, ability to inhibit airway smooth muscle cell migration or proliferation, or ability to influence development of airway hyper-reactivity during allergen challenge.

27. A method for decreasing airway remodeling in a subject, comprising administering to a subject in need thereof an effective amount of a composition comprising a sema3E polypeptide, wherein the subject has decreased airway remodeling when compared to the subject before the administering.

28. A method for treating asthma in a subject, comprising administering to the subject an effective amount of a composition comprising a sema3E polypeptide.

29. A method for treating a subject having, or at risk of having, acute asthma, comprising:
administering to a subject in need thereof a composition comprising sema3E polypeptide, wherein the subject has decreased airway resistance, decreased tissue resistance, decreased lung elastance, decreased eosinophilia in the bronchoalveolar space, or a combination thereof, when compared to the subject before the administering.

30. A method for reducing inflammation of a subject's airway, comprising:
administering to a subject in need thereof a composition comprising sema3E polypeptide, wherein the subject has decreased inflammation of the airway when compared to the subject before the administering.

31. A method for decreasing proliferation of a cell, comprising contacting an airway smooth muscle cell with an effective amount of a composition comprising a sema3E polypeptide.

32. A method for decreasing migration of a cell, comprising contacting an airway smooth muscle cell with an effective amount of a composition comprising a sema3E polypeptide.

33. The method of any of claims 27-30 wherein the subject is a human.

34. The method of claim 31 or 32 wherein the cell is a human cell.

35. The method of any of claims 27-30 wherein the method further comprises administering a therapeutic compound.

36. The method of claim 35 wherein the therapeutic compound is selected from an inhaled corticosteroid, an oral corticosteroid, a bronchodilator, a leukotriene antagonist, and an antihistamine.

37. The method of claim 27 wherein the subject has, or at risk of having, an inflammatory airway disease.

38. The method of claim 37 wherein the inflammatory airway disease is selected from acute asthma and chronic asthma.

39. The method of claim 31 or 32 wherein the cell is ex vivo.

40. The method of claim 31 or 32 wherein the cell is in vivo.

41. The method of any of claims 27-32 wherein the sema3E polypeptide comprises a KRRXRR consensus site, wherein X is any amino acid.

42. The method of claim 41 wherein the sema3E polypeptide further comprises an RXXR consensus site, wherein X is any amino acid.

43. The method of claim 42 wherein the sema3E polypeptide further comprises a second RXXR consensus site, wherein X is any amino acid.

44. The method of any of claims 27-32 wherein the sema3E polypeptide comprises a Sema domain.

45. The method of claim 44 wherein the sema3E polypeptide further comprises one or more domains selected from a cystine rich domain, an immunoglobulin domain, and a short basic domain.

46. The use of claim 44 wherein the sema3E polypeptide further comprises a cystine rich domain and an immunoglobulin domain.

47. The method of any of claims 27-32 wherein the sema3E polypeptide comprises an amino acid sequence having at least 80% identity with SEQ ID NO:2.

48. The method of any of claims 27-32 wherein the sema3E polypeptide is a fusion polypeptide.

49. The method of any of claims 27-32 wherein the sema3E polypeptide has activity when determined by ability to bind to Plexin D1, ability to inhibit airway smooth muscle cell migration or proliferation, or ability to influence development of airway hyper-reactivity during allergen challenge.

50. A method for decreasing airway remodeling in a subject, comprising administering to a subject in need thereof an effective amount of a composition comprising a sema3E polynucleotide, wherein the subject has decreased airway remodeling when compared to the subject before the administering, and wherein the sema3E polynucleotide encodes a sema3E polypeptide.

51. A method for treating asthma in a subject, comprising administering to the subject an effective amount of a composition comprising a sema3E polynucleotide, wherein the sema3E polynucleotide encodes a sema3E polypeptide.

52. A method for treating a subject having, or at risk of having, acute asthma, comprising:
administering to a subject in need thereof a composition comprising sema3E polynucleotide, wherein the subject has decreased airway resistance, decreased tissue resistance, decreased lung elastance, decreased eosinophilia in the bronchoalveolar space, or a combination thereof, when compared to the subject before the administering, wherein the sema3E polynucleotide encodes a sema3E polypeptide.

53. A method for reducing inflammation of a subject's airway, comprising:
administering to a subject in need thereof a composition comprising sema3E polynucleotide, wherein the subject has decreased inflammation of the airway when compared to the subject before the administering, wherein the sema3E polynucleotide encodes a sema3E polypeptide.

54. A method for decreasing proliferation of a cell, comprising contacting an airway smooth muscle cell with an effective amount of a composition comprising a sema3E polynucleotide, wherein the sema3E polynucleotide encodes a sema3E polypeptide.

55. A method for decreasing migration of a cell, comprising contacting an airway smooth muscle cell with an effective amount of a composition comprising a sema3E polynucleotide, wherein the sema3E polynucleotide encodes a sema3E polypeptide.

56. The method of any of claims 50-53 wherein the subject is a human.

57. The method of claim 54 or 55 wherein the cell is a human cell.

58. The method of any of claims 50-53 wherein the method further comprises administering a therapeutic compound.

59. The method of claim 58 wherein the therapeutic compound is selected from an inhaled corticosteroid, an oral corticosteroid, a bronchodilator, a leukotriene antagonist, and an antihistamine.

60. The method of claim 50 wherein the subject has, or at risk of having, an inflammatory airway disease.

61. The method of claim 60 wherein the inflammatory airway disease is selected from acute asthma and chronic asthma.

62. The method of claim 54 or 55 wherein the cell is ex vivo.

63. The method of claim 54 or 55 wherein the cell is in vivo.

64. The method of any of claims 27-32 wherein the sema3E polypeptide comprises a KRRXRR consensus site, wherein X is any amino acid.

65. The method of claim 64 wherein the sema3E polypeptide further comprises an RXXR consensus site, wherein X is any amino acid.

66. The method of claim 65 wherein the sema3E polypeptide further comprises a second RXXR consensus site, wherein X is any amino acid.

67. The method of any of claims 50-55 wherein the sema3E polypeptide comprises a Sema domain.

68. The method of claim 67 wherein the sema3E polypeptide further comprises one or more domains selected from a cystine rich domain, an immunoglobulin domain, and a short basic domain.

69. The method of claim 67 wherein the sema3E polypeptide further comprises a cystine rich domain and an immunoglobulin domain.

70. The method of any of claims 50-55 wherein the sema3E polypeptide comprises an amino acid sequence having at least 80% identity with SEQ ID NO:2.

71. The method of any of claims 50-55 wherein the sema3E polypeptide is a fusion polypeptide.

72. The method of any of claims 50-55 wherein the sema3E polynucleotide is present in a vector.

73. The use of claim 72 wherein the vector is a viral vector.

74. The method of any of claims 50-55 wherein the sema3E polypeptide has activity when determined by ability to bind to Plexin D1, ability to inhibit airway smooth muscle cell migration or proliferation, or ability to influence development of airway hyper-reactivity during allergen challenge.

75. A method for diagnosing whether a subject has asthma, comprising:
obtaining a biological sample from the subject, wherein the biological sample comprises an airway smooth muscle cell;
measuring the expression of a sema3E polypeptide by the airway smooth muscle cell; and
comparing the expression of a sema3E polypeptide by the airway smooth muscle cell with a control cell, wherein the presence of an airway smooth muscle cell that has reduced expression of a sema3E polypeptide compared to a control cell indicates the subject has asthma.

76. A method for evaluating treatment options for a subject having asthma comprising:
obtaining a biological sample from the subject, wherein the biological sample comprises an airway smooth muscle cell;
measuring the expression of a sema3E polypeptide by the airway smooth muscle cell; and
comparing the expression of a sema3E polypeptide by the airway smooth muscle cell with a control cell, wherein the presence of an airway smooth muscle cell that has reduced expression of a sema3E polypeptide compared to a control cell indicates the subject may be treated with a sema3E polypeptide.

77. A method for diagnosing whether a subject has asthma, comprising:
obtaining a biological sample from the subject;
measuring the level of sema3E polypeptide in the biological sample; and
comparing the level of sema3E polypeptide in the biological sample with the level of sema3E polypeptide in a control biological sample obtained from a healthy subject, wherein the presence of a decreased level of sema3E polypeptide compared to the control biological sample indicates the subject has asthma.

78. A method for evaluating treatment options for a subject having asthma comprising:
obtaining a biological sample from the subject;
measuring the level of sema3E polypeptide in the biological sample; and
comparing the level of sema3E polypeptide in the biological sample with the level of sema3E polypeptide in a control biological sample obtained from a healthy subject, wherein the presence of a decreased level of sema3E polypeptide compared to the control biological sample indicates the subject may be treated with a sema3E polypeptide.

79. The method of any of claim 71-74 wherein the biological sample comprises bronchial tissue, tracheal tissue, broncholaveolar lavage, sputum or serum.

80. The method of any of claim 71-74 further comprising administering to the subject a sema3E polypeptide or a fragment thereof.

81. The method of any of claim 71-74 wherein the subject is a human.

82. A method for identifying an agent that increases sema3E polypeptide expression in a cell, the method comprising:
contacting an airway smooth muscle cell with an agent;
measuring the expression of semaphorin 3E by the airway smooth muscle cell, wherein an increase in expression of semaphorin 3E in an airway smooth muscle cell compared to a control cell that was not contacted with the agent indicates the agent increases semaphorin 3E expression in a cell.

83. The method of claim 78 wherein the expression of sema3E polypeptide is determined by measuring sema3E polypeptide.

84. The method of claim 78 wherein the expression of sema3E polypeptide is determined by measuring a polynucleotide encoding the sema3E polypeptide.
